SEQUENCE LISTING (7 sequences)

SEQ ID NO: 1 - protein, 30 aa, synthetic construct
GRSLPTCLPVCGLPKFSRKLMARIFNGRPA

SEQ ID NO: 2 - protein, 5 aa, synthetic construct
KLMAR

SEQ ID NO: 3 - protein, 30 aa, Homo sapiens
GRSLPTCLPECGQPSRSLPSLVKRIIGGRN

SEQ ID NO: 4 - protein, 5 aa, synthetic construct
DDDDK

SEQ ID NO: 5 - protein, 30 aa, Homo sapiens
VDCRAPGELEHGLITFSTRNNLTTYKSEIK

SEQ ID NO: 6 - protein, 30 aa, Homo sapiens
STRNNLTTYKSEIKYSCQEPYYKMLNNNTG

SEQ ID NO: 7 - protein, 30 aa, Homo sapiens
GELEHGLITFSTRNNLTTYKSEIKYSCQEP

(19) United States
(12) Patent Application Publication
(10) Pub. No.: US 2025/0304939 A1
(43) Pub. Date: Oct. 2, 2025

(54) TAILORED ANTI-FUNGAL DRUGS AND TOOLS TO DIAGNOSE INVASIVE FUNGAL INFECTIONS

(71) Applicant: RIGSHOSPITALET, Copenhagen Ø (DK)

(72) Inventors: Anne ROSBJERG, Copenhagen Ø (DK); Mikkel-Ole SKJØDT, Copenhagen Ø (DK); Peter GARRED, Copenhagen Ø (DK); Rafael BAYARRI-OLMOS, Copenhagen Ø (DK)

(73) Assignee: RIGSHOSPITALET, Copenhagen Ø (DK)

(21) Appl. No.: 18/881,985

(22) PCT Filed: Jul. 6, 2023
(86) PCT No.: PCT/EP2023/068738

§ 371 Date: Jan. 7, 2025

(30) Foreign Application Priority Data
Jul. 7, 2022 (EP) 22183703.2

(51) Int. Cl. C12N 9/64; A61K 38/00; A61K 47/64; A61P 31/10

(52) U.S. Cl. CPC C12N 9/6424; A61K 47/64; A61P 31/10; A61K 38/00; C07K 2319/30

(57) ABSTRACT

The present application presents peptides that binds selectively to fungi, in particular opportunistic fungi, and not to human cells. These properties can be utilized to specifically target invading fungal pathogens, and via genetic engineering create peptibodies. Thus, the present application describes novel approaches for detection and treatment of infections caused by fungi.

### FIELD

The present application presents peptides that binds selectively to fungi, in particular opportunistic fungi, and not to human cells. These properties can be utilized to specifically target invading fungal pathogens, and via genetic engineering create peptibodies. Thus, the present application describes novel approaches for detection and treatment of infections caused by fungi.

The specific sequence could be a tool for targeted treatment of fungal infections. The sequence can be coupled to biological active components that provide a function to the peptide and that contributes to the physiochemical properties of the construct.

### BACKGROUND

Fungal infections represent an increasing health problem worldwide. Patients undergoing high dose chemotherapy or stem cell/organ transplantation are at high risk of being infected with opportunistic fungal pathogens. Omnipresent, airborne fungal spores can reach the lungs and cause invasive infections. Despite prophylactic antifungal treatment, fungal infections still constitute a severe clinical problem associated with high mortality rates (30-90%). The problem is aggravated by the fact that drug-resistant strains of one of the dominating pathogens, Aspergillus fumigatus, appear more and more frequently.

Moreover, the fungicidal compounds currently used in the clinic have the disadvantage of making drug-drug interactions and giving rise to adverse and often systemic toxic reactions in the patient.

Hence, to develop a targeted drug delivery system that circumvents systemic exposure and circumvents the increasing resistance problem is a desirable goal, as there is an unmet need in the treatment of opportunistic fungal infections.

### SUMMARY

This disclosure presents peptides that binds selectively to fungi, and which does not bind to human cells.

In one aspect, the present disclosure relates to an isolated fungus binding amino acid comprising a binding part having at least 70% sequence identity to SEQ ID NO: 1.

In another aspect, the present disclosure relates to an isolated fungus binding amino acid comprising a binding motif having at least 80% sequence identity to SEQ ID NO: 2 In another aspect, the present disclosure relates to fusion-protein constructs that contain the amino acid sequence as described herein and the amino acid sequence of an Fc-region from an immunoglobulin.

In another aspect, the present disclosure relates to a fungus binding peptibody comprising a fungus binding amino acid conjugated to an Fc-region from an immunoglobulin.

In another aspect, the present disclosure relates to an isolated nucleic acid encoding a fungus binding amino acid sequence as described herein or a nucleic acid construct encoding a fusion-protein as described herein.

In another aspect, the present disclosure relates to a host cell comprising a nucleic acid or nucleic acid construct as described herein.

In another aspect, the present disclosure relates to a method for producing a fungus binding amino acid sequence, the method comprising
culturing the host cell as described herein, and
recovering the fungus binding amino acid sequence.

As the skilled addressee knows, then the fungus binding amino acid sequence can also be made via chemical synthesis of peptides. Chemical synthesis of peptides can be carried out using classical solution-phase techniques, although these have been replaced in most research and development settings by solid-phase methods. However, solution-phase synthesis retains its usefulness in large-scale production of peptides for industrial purposes.

In another aspect, the present disclosure relates to a composition comprising a fungus binding amino acid sequence as described herein, and the use of such as a medicament, in therapy, prophylaxis and diagnostics.

In another aspect, the present disclosure relates to a method for detecting a fungus in a sample comprising
providing a sample suspected of containing a fungus
adding a fungus binding amino acid as described herein to the sample
identifying the binding of the fungus binding amino acid to the fungus or fungal fragments within the sample.

The compound can also be used to deliver site-directed anti-fungal compounds.

Another application for the peptide is in diagnostics. Invasive fungal infections are not easily diagnosed and often require several attempts using ELISA, PCR, cultivation, x-rays, CT or MRI scanning. The currently used assays lack sensitivity and new diagnostic tools are urgently needed. Our peptide would be a fast way to detect whether the patient has a fungal infection using plasma or lung fluid or by in vivo imaging.

### DETAILED DESCRIPTION

As shown in the Examples below MASP-1, a serine protease from the complement system, binds directly to various pathogenic fungi, for example, Aspergillus fumigatus. This discovery was utilized to design a targeted antifungal compound comprising the specific fungus binding amino acid sequences as disclosed herein.

MASP-1 binding was tested on the different growth stages of A. fumigatus. First, the A. fumigatus conidia were incubated on microscopy glass slides for 0, 4, 8 and 16 hours to obtain resting conidia, swollen conidia, germ tubes and hyphae. Recombinant MASP-1 was then added in a concentration of 5 μg/ml and binding was detected with a pan anti-MASP-1/-3/MAP-1 monoclonal antibody 8B3 and Alexa fluor 488-coupled goat anti-mouse antibody. Using fluorescence microscopy, recombinant MASP-1 binding was detected for all growth stages (FIG. 1-8). This is very surprising since until now, MASP-1 is known only to interact with endogenous pattern recognition molecules (PRMs) and not so-called pathogen-associated molecular patterns (PAMPs). As exemplified below, then FIGS. 13-15 demonstrate binding to the species Lichtemia corymbifera, Mucor circinelloides and Rhizopus arrhizus all belonging to the Mucorales order.

The novel aspect and the surprise element of the research disclosed herein lie in the unforeseen interaction of serine protease MASP-1, a protein typically implicated in the complement and coagulation systems, with the fungal species Aspergillus fumigatus. This previously unknown binding inspired a comprehensive exploration of its biological significance and possible dependence on the protein's activation state.

MASP-1 is produced as an inactive zymogen that undergoes activation during an immune response. Consequently, its interaction with Aspergillus fumigatus could be contingent on the state of the protein—active or inactive. To investigate this, we experimented with both the zymogen form of wild-type recombinant MASP-1 (rMASP-1 (444KLMAR448)) and the zymogen and active forms of a mutated variant (rMASP-1 (444DDDDK448)).

Unexpectedly, this investigation revealed that the mutated rMASP-1 (444DDDDK448) failed to bind to Aspergillus fumigatus conidia, regardless of the protein's activation state. In contrast, the wild-type rMASP-1 (444KLMAR448) demonstrated binding, emphasizing that the key determinant for the interaction was the alteration of the amino acid sequence rather than the protein's state of activation (see FIG. 28).

In an unanticipated twist, the investigation into whether MASP-1's binding was state-dependent led to the unexpected discovery of a specific amino acid sequence essential for the fungus-MASP interaction. This finding has been instrumental in pinpointing the specific part of MASP-1 involved in interactions with Aspergillus fumigatus. It is this surprising revelation that underscores the novelty and significance of the findings disclosed herein, setting the foundation for further research into this unexpected fungal-protein interaction.

### Fungus Binding Amino Acid

In the present context, a fungus binding peptide relates to a peptide that binds to fungi belonging to the order of Mucorales, and in particular the genus Aspergillus.

Thus, in one or more exemplary embodiments, the present disclosure relates to an isolated fungus binding amino acid comprising a binding part having at least 70% sequence identity to SEQ ID NO: 1.

The ability of such a peptide to bind to a fungus is exemplified for instance in FIGS. 9-16 by a 30 amino acid peptide according to SEQ ID NO: 1 that binds Aspergillus fumigatus.

### Sequence Identity

In the present context, proteins, homologues, derivatives, peptides and/or fragments thereof having an amino acid sequence at least, for example 70% identical to a reference amino acid sequence, is intended that the amino acid sequence of e.g., the peptide is identical to the reference sequence, except that the amino acid sequence may include up to 30 mutations per each 100 amino acids of the reference amino acid sequence.

In other words, to obtain a sequence at least 30% identical to a reference sequence, up to 30% of the amino acids or nucleotides in the reference sequence may be deleted or substituted with another amino acid/nucleotide, or several amino acids/nucleotides up to 30% of the total amount of the reference sequence.

These mutations of the reference sequence may occur at the amino or carboxy terminal positions of the reference amino acid sequence or anywhere between those terminal positions, interspersed either individually among amino acids in the reference sequence or in one or more contiguous groups within the reference sequence.

Methods to determine identity and similarity are codified in publicly available programs. Preferred computer program methods to determine identity and similarity between two sequences include, but are not limited to, the GCG program package, BLASTP, BLASTN, and FASTA.

The BLASTX program is publicly available from NCBI and other sources. Each sequence analysis program has a default scoring matrix and default gap penalties. In general, a molecular biologist would be expected to use the default settings established by the software program used.

### Amino Acid Sequence Identity

Thus, in one or more exemplary embodiments, the present disclosure relates to an isolated fungus binding amino acid comprising a binding part having at least 70% sequence identity to SEQ ID NO: 1, such as for example 71% sequence identity to SEQ ID NO: 1, 72% sequence identity to SEQ ID NO: 1, 73% sequence identity to SEQ ID NO: 1, 74% sequence identity to SEQ ID NO: 1, 75% sequence identity to SEQ ID NO: 1, 76% sequence identity to SEQ ID NO: 1, 77% sequence identity to SEQ ID NO: 1, 78% sequence identity to SEQ ID NO: 1, 79% sequence identity to SEQ ID NO: 1, 80% sequence identity to SEQ ID NO: 1, 81% sequence identity to SEQ ID NO: 1, 82% sequence identity to SEQ ID NO: 1, 83% sequence identity to SEQ ID NO: 1, 84% sequence identity to SEQ ID NO: 1, 85% sequence identity to SEQ ID NO: 1, 86% sequence identity to SEQ ID NO: 1, 87% sequence identity to SEQ ID NO: 1, 88% sequence identity to SEQ ID NO: 1, 89% sequence identity to SEQ ID NO: 1, 90% sequence identity to SEQ ID NO: 1, 91% sequence identity to SEQ ID NO: 1, 92% sequence identity to SEQ ID NO: 1, 93% sequence identity to SEQ ID NO: 1, 94% sequence identity to SEQ ID NO: 1, 95% sequence identity to SEQ ID NO: 1, 96% sequence identity to SEQ ID NO: 1, 97% sequence identity to SEQ ID NO: 1, 98% sequence identity to SEQ ID NO: 1, 99% sequence identity to SEQ ID NO: 1 or 100% sequence identity to SEQ ID NO: 1.

### Fungus Binding Peptide Motif

Peptide motifs are amino acid patterns that are often conserved between functional peptide homologs, and which define structural peptide elements that are important for the specific functionality or bioactivity of a given polypeptide. Therefore, in the present context a fungus binding peptide motif is a motif that is required for a polypeptide or polypeptide construct to be able to bind a fungal cell or a fungal fragment.

The fungus binding activity of SEQ ID NO: 1—a MASP-1 peptide fragment of 30 amino acids—tagged with biotin was demonstrated to bind several Aspergillus species by incubation together with several Aspergillus subspecies and detected by flow cytometry using FITC-coupled streptavidin as shown in FIGS. 9-12.

Additionally, SEQ ID NO: 1 tagged with biotin also demonstrated binding to the species Lichtemia crymbifera, Mucor circinelloides and Rhizopus arrhizus all belonging to the Mucorales order. Binding was detected by flow cytometry using FITC-coupled streptavidin as shown in FIGS. 13-15.

Another 30 amino acid sequence from the CUB1 domain and a random 30 amino acid peptide were both tagged with biotin and compared to the biotin tagged MASP-1 peptide according to SEQ ID NO: 1 for fungus binding activity in a flow cytometry assay using FITC-coupled streptavidin. However, only the MASP-1 peptide according to SEQ ID NO: 1 showed fungus binding activity (FIGS. 16-18).

In an effort to identify the peptide motif responsible for the fungus binding activity of MASP-1, i.e. the fungus binding peptide motif, the MASP-1 wild type sequence was mutated so that a 5 amino acid section ⁴⁴⁴KLMAR⁴⁴⁸ was substituted by the 5 amino acid section DDDDK.

The fungus binding ability of the wild type MASP-1 peptide and the DDDDK substituted MASP-1 was assayed by comparing binding of 5 μg/ml of each peptide to conidia from A. fumigatus. As shown in FIG. 26, The peptide having the KLMAR sequence has fungus binding activity while the mutated peptide with the DDDDK substitution does not bind to conidia of the tested fungi.

This result highlights the importance of the 5 amino acid fungus binding peptide motif KLMAR [SEQ ID NO: 2] for a peptide to possess fungus binding activity.

Therefore, in the present context, a fungus binding peptide motif is a motif that binds a fungal cell or fungal cell fragment via fluorescence microscopy using for example a secondary antibody, as exemplified below.

Thus, in one or more exemplary embodiments, the present disclosure relates to any fungus binding peptide motif that binds a fungal cell or fungal cell fragment.

Thus, in one or more exemplary embodiments, the present disclosure relates to a fungus binding peptide motif comprising a binding motif having at least 80% sequence identity to SEQ ID NO: 2.

### Motif Sequence Identity

In line with the above defined sequence identity, the binding motif may vary, thus in one or more exemplary embodiments, the binding motif has at least 80% sequence identity to SEQ ID NO: 2, such as for example 81% sequence identity to SEQ ID NO: 2, 82% sequence identity to SEQ ID NO: 2, 83% sequence identity to SEQ ID NO: 2, 84% sequence identity to SEQ ID NO: 2, 85% sequence identity to SEQ ID NO: 1, 86% sequence identity to SEQ ID NO: 2, 87% sequence identity to SEQ ID NO: 2, 88% sequence identity to SEQ ID NO: 2, 89% sequence identity to SEQ ID NO: 2, 90% sequence identity to SEQ ID NO: 2, 91% sequence identity to SEQ ID NO: 2, 92% sequence identity to SEQ ID NO: 2, 93% sequence identity to SEQ ID NO: 2, 94% sequence identity to SEQ ID NO: 1, 95% sequence identity to SEQ ID NO: 2, 96% sequence identity to SEQ ID NO: 2, 97% sequence identity to SEQ ID NO: 2, 98% sequence identity to SEQ ID NO: 2, 99% sequence identity to SEQ ID NO: 2 or 100% sequence identity to SEQ ID NO: 2.

In one or more exemplary embodiments, the present disclosure relates to a fungus binding peptide motif that is at least 7 amino acids long and having at least 80% sequence identity to SEQ ID NO: 2.

In one or more exemplary embodiments, the fungus binding peptide motif relates to an isolated amino acid sequence comprising at least 7 amino acids and wherein said amino acid sequence includes a section of 5 amino acids having at least 80% sequence identity to SEQ ID NO: 2.

In one or more exemplary embodiments, the fungus binding peptide motif relates to an isolated amino acid sequence comprising at least 7 amino acids and wherein said amino acid sequence includes a section of 5 consecutive amino acids having at least 80% sequence identity to SEQ ID NO: 2.

In one or more exemplary embodiments, the isolated fungus binding amino acid sequence is at least 10 amino acids long and comprise a binding motif having at least 80% sequence identity to SEQ ID NO: 2.

Thus, in one or more exemplary embodiments, the fungus binding peptide motif relates to an isolated amino acid sequence comprising at least 10 amino acids, and wherein said amino acid sequence includes a section consisting of 5 amino acids having at least 80% sequence identity to SEQ ID NO: 2.

Thus, in one or more exemplary embodiments, the isolated fungus binding peptide motif relates to an isolated amino acid sequence comprising at least 10 amino acids, and wherein said amino acid sequence includes a section consisting of 5 consecutive amino acids having at least 80% sequence identity to SEQ ID NO: 2.

In one or more exemplary embodiments, the present disclosure relates to an isolated fungus binding peptide motif that is at least 15 amino acids long and having at least 80% sequence identity to SEQ ID NO: 2.

In one or more exemplary embodiments, the fungus binding peptide motif relates to an isolated amino acid sequence comprising at least 15 amino acids and wherein said amino acid sequence includes a section of 5 amino acids having at least 80% sequence identity to SEQ ID NO: 2.

In one or more exemplary embodiments, the fungus binding peptide motif relates to an isolated amino acid sequence comprising at least 15 amino acids and wherein said amino acid sequence includes a section of 5 consecutive amino acids having at least 80% sequence identity to SEQ ID NO: 2.

In one or more exemplary embodiments, the present disclosure relates to an isolated fungus binding peptide motif that is at least 20 amino acids long and having at least 80% sequence identity to SEQ ID NO: 2.

In one or more exemplary embodiments, the fungus binding peptide motif relates to an isolated amino acid sequence comprising at least 20 amino acids and wherein said amino acid sequence includes a section of 5 amino acids having at least 80% sequence identity to SEQ ID NO: 2.

In one or more exemplary embodiments, the fungus binding peptide motif relates to an isolated amino acid sequence comprising at least 20 amino acids and wherein said amino acid sequence includes a section of 5 consecutive amino acids having at least 80% sequence identity to SEQ ID NO: 2.

In one or more exemplary embodiments, the present disclosure relates to an isolated fungus binding peptide motif that is at least 25 amino acids long and having at least 80% sequence identity to SEQ ID NO: 2.

In one or more exemplary embodiments, the fungus binding peptide motif relates to an isolated amino acid sequence comprising at least 25 amino acids and wherein said amino acid sequence includes a section of 5 amino acids having at least 80% sequence identity to SEQ ID NO: 2.

In one or more exemplary embodiments, the fungus binding peptide motif relates to an isolated amino acid sequence comprising at least 25 amino acids and wherein said amino acid sequence includes a section of 5 consecutive amino acids having at least 80% sequence identity to SEQ ID NO: 2.

In one or more exemplary embodiments, the present disclosure relates to an isolated fungus binding peptide motif that is at least 30 amino acids long and having at least 80% sequence identity to SEQ ID NO: 2.

In one or more exemplary embodiments, the fungus binding peptide motif relates to an isolated amino acid sequence comprising at least 30 amino acids and wherein said amino acid sequence includes a section of 5 amino acids having at least 80% sequence identity to SEQ ID NO: 2.

In one or more exemplary embodiments, the fungus binding peptide motif relates to an isolated amino acid sequence comprising at least 30 amino acids and wherein said amino acid sequence includes a section of 5 consecutive amino acids having at least 80% sequence identity to SEQ ID NO: 2.

In one or more exemplary embodiments, a fungus binding peptide motif as disclosed herein comprises an isolated fungus binding peptide motif having at least 60% sequence identity to an amino acid sequence according to SEQ ID NO: 2.

### Fungi

The fungus binding amino acids and fungus binding peptide motifs disclosed herein binds selectively to fungi, in particular opportunistic pathogenic fungi, including fungi belonging to the order of Mucorales and the species Aspergillus.

Thus, as these fungi are all relevant from clinical perspective, The fungus binding amino acids and fungus binding peptide motifs disclosed herein could be used a tool for example in targeted treatment or diagnosis of fungal infections.

The fungus binding amino acids or motifs can be coupled to biological active components that provide a function to the peptide and that contributes to the physiochemical properties of the construct.

In the present context, a fungus is a eukaryotic organism that includes microorganisms such as yeasts and molds, as well as the more familiar mushrooms.

The fungal life cycle for most fungi and in particular for the filamentous fungi can be divided into four growth stages, resting conidia (spores), swollen conidia, germ tubes and hyphae. The isolated fungus binding amino acid and fungus binding peptide motifs disclosed herein binds to all these four fungal growth stages as shown in FIGS. 1-8.

The binding of Peptibody to A. fumigatus conidia was tested by incubating 10 μg/ml of purified Peptibody with 1×10⁷ heat-inactivated or live conidia/ml and the binding was detected with a rabbit anti-human IgG antibody and a FITC-coupled goat anti-rabbit antibody using flow cytometry. As shown in FIG. 27, the Peptibody binds to both the heat-inactivated and live A. fumigatus conidia.

A. fumigatus is a filamentous fungus, which means that the conidia grow into elongated structures called germ tubes and hyphae. The fungus expands in its natural environment by growing hyphae structures and these growth stages are also found in infected patients. Thus, binding to these growth stages is likely an important feature of an antifungal drug in case the fungus has already developed in the patient prior to diagnosis and treatment. Binding of the Peptibody to germ tubes and hyphae was tested using fluorescence microscopy. First, the A. fumigatus conidia were incubated on microscopy glass slides for 6 and 18 hours to develop germ tubes and hyphae. Afterward, the Peptibody was added in a concentration of 5 or 10 μg/ml and binding was detected with rabbit anti-human IgG antibody and an Alexa fluor 488-coupled goat anti-rabbit antibody. The Peptibody binding was detected on both germ tubes and hyphae as shown in FIGS. 20-22.

In one or more exemplary embodiments, the fungus binding amino acids and fungus binding peptide motifs disclosed herein binds selectively to resting conidia (spores).

In one or more exemplary embodiments, the fungus binding amino acids and fungus binding peptide motifs disclosed herein binds selectively to swollen conidia.

In one or more exemplary embodiments, the fungus binding amino acids and fungus binding peptide motifs disclosed herein binds selectively to germ tubes.

In one or more exemplary embodiments, the fungus binding amino acids and fungus binding peptide motifs disclosed herein binds selectively to hyphae.

In one or more exemplary embodiments, the fungus is a cellular organism belonging to the order of the Mucorales.

Mucorales is the largest and best studied order of zygomycete fungi. Members of this order are sometimes called pin molds. The term mucormycosis is commonly used for infections caused by molds belonging to the order Mucorales.

Thus in one or more exemplary embodiments the present disclosure relates to a fungus belonging to any one of the genus selected from the list consisting of Actinomucor spp, Apophysomyces spp, Benjaminella spp., chaetocladium spp., Circinella spp., Cokeromyces spp., Dicranophora spp., Ellisomyces spp., Helicostylum spp., Hyphomucor spp., Kirkomyces spp., Mucor spp., Parasitella spp., Pilaira spp., Pliphora spp., Pirella spp., Rhizomucor spp., Lichtheimia spp., Rhizopodopsis spp., Rhizopus spp., Sporodinella spp., Syzygites spp., Thamnidium spp., Thermomucor spp., Zygorhynchus spp., Fusarium spp., Scedosporium spp., Penicillium spp., Aspergillus spp., Candida spp., and Cryptococcus spp.

In one or more exemplary embodiments, the present disclosure relates to a fungus belonging to any one of the genus Rhizopus spp., Mucor spp., and Lichtemia spp.

In one or more exemplary embodiments, the present disclosure relates to a fungus belonging to the genus Rhizopus spp.

In one or more embodiments the fungus belonging to the genus Rhizopus spp. is Rhizopus arrhizus.

In one or more exemplary embodiments, the present disclosure relates to a fungus belonging to the genus Mucor spp.

In one or more embodiments the fungus belonging to the genus Mucor spp. is Mucor circinelloides.

In one or more exemplary embodiments, the present disclosure relates to a fungus belonging to the genus Lichtemia.

In one or more exemplary embodiments the fungus belonging to the genus Lichtemia spp. is Lichtemia corymbifera.

In one or more exemplary embodiments the present disclosure relates to a fungus belonging to any of the genus Aspergillus spp., Candida spp., and Cryptococcus spp.

In one or more exemplary embodiments the present disclosure relates to a fungus belonging to the genus Aspergillus spp.

In one or more exemplary embodiments of the present disclosure, the fungus belonging to the genus Aspergillus, is any one of the species selected from the list consisting of Aspergillus fumigatus, Aspergillus flavus, Aspergillus terreus, Aspergillus niger and Aspergillus nidulans.

In one or more exemplary embodiments of the present disclosure, the fungus belonging to the genus Aspergillus, is Aspergillus fumigatus.

In one or more exemplary embodiments of the present disclosure, the fungus belonging to the genus Aspergillus, is Aspergillus flavus.

In one or more exemplary embodiments of the present disclosure, the fungus belonging to the genus Aspergillus, is Aspergillus terreus.

In one or more exemplary embodiments of the present disclosure, the fungus belonging to the genus Aspergillus, is Aspergillus niger.

In one or more exemplary embodiments of the present disclosure, the fungus belonging to the genus Aspergillus, is Aspergillus nidulans.

In one or more exemplary embodiments the present disclosure relates to a fungus belonging to the genus Candida spp.

In one or more exemplary embodiments, the fungus binding amino acid does not bind to a fungus belonging to the genus Candida spp.

In one or more exemplary embodiments the present disclosure relates to a fungus belonging to the genus Cryptococcus spp.

In one or more exemplary embodiments, the fungus binding amino acid does not bind to a fungus belonging to the genus Cryptococcus spp.

### Opportunistic Fungal Pathogens

Opportunistic fungal pathogens are fungi that are nonpathogenic in the immunocompetent host. These fungi may cause opportunistic infections in weakened or immunocompromised hosts. Such fungi can for example be part of the upper respiratory tract flora and may cause pulmonary infection in hosts that become weakened or immunocompromised because of a different condition or disorder.

In one or more exemplary embodiments, the isolated fungus binding amino acid or motifs binds to an opportunistic fungus.

Opportunistic fungal infections are commonly caused by Aspergillus spp., Candida spp., Cryptococcus spp. with Aspergillus being the mold most associated with infection in patients with hematological malignancy and bone marrow disease.

Thus, in one or more exemplary embodiments, the opportunistic fungus is selected from the group consisting of Aspergillus spp., Candida spp., and Cryptococcus spp.

In one or more exemplary embodiments, the opportunistic fungus belongs to the Aspergillus genus.

In one or more exemplary embodiments of the present disclosure, the fungus belonging to the genus Aspergillus, is any one of the species selected from the list consisting of Aspergillus fumigatus, Aspergillus flavus, Aspergillus terreus, Aspergillus niger and Aspergillus nidulans.

In one or more exemplary embodiments of the present disclosure, the fungus belonging to the genus Aspergillus, is Aspergillus fumigatus.

In one or more exemplary embodiments of the present disclosure, the fungus belonging to the genus Aspergillus, is Aspergillus flavus.

In one or more exemplary embodiments of the present disclosure, the fungus belonging to the genus Aspergillus, is Aspergillus terreus.

In one or more exemplary embodiments of the present disclosure, the fungus belonging to the genus Aspergillus, is Aspergillus niger.

In one or more exemplary embodiments of the present disclosure, the fungus belonging to the genus Aspergillus, is Aspergillus nidulans.

In one or more exemplary embodiments, the opportunistic fungus belongs to the Candida genus.

In one or more exemplary embodiments, the opportunistic fungus belongs to the Cryptococcus genus.

In one or more exemplary embodiments, the opportunistic fungus is Aspergillus fumigatus.

### Fungal Fragments

Fungi may reproduce asexually by fragmentation, budding, or producing spores. Fragments of hyphae can grow new colonies. Mycelial fragmentation occurs when a fungal mycelium separates into pieces with each component growing into a separate mycelium. Fungal fragments and undocumented conidia may function as aeroallergen sources.

Thus, in one or more exemplary embodiments the isolated fungus binding amino acids and fungus binding peptide motifs disclosed herein binds to fungal fragments. Within the context of the present disclosure a fungal fragment is fragments of fungal conidia, fungal germ tubes or fungal hyphae.

### Fusion Proteins

Fusion proteins are proteins created through the joining of genetic sequences encoding for partial or whole sequence of different proteins. Such joining of genetic sequences results in the transcription and translation of a single continuous genetic sequence, thereby resulting in chimeric proteins comprising the protein equivalents of each of the genetic sequences are fused to each other through peptide bonds as part of the translation process.

The peptide-coupled functional component, e.g., an immunoglobulin Fc region, can work therapeutically against fungal infections by recruiting the immune apparatus of the patient, thereby facilitating clearance of the pathogen. An Fc region enables fungal killing via phagocytosis, antibody-dependent cellular cytotoxicity and by activating the complement system mediating further opsonization and phagocytosis as well as anaphylatoxin release and recruitment of inflammatory cells and complement-mediated cytolysis. Gathering multiple peptides with Fc regions also enables agglutination, which can block the fungi from accessing the epithelium and contribute to fungal clearance by making the phagocytosis more efficient.

Thus, in one or more exemplary embodiments, the present disclosure relates to a fusion-protein construct that contains a fungus binding amino acid as disclosed herein and the amino acid sequence of an Fc-region.

In one or more exemplary embodiments, the present disclosure relates to a fusion-protein construct that contains a fungus binding amino acid having at least 70% sequence identity to SEQ ID NO: 1 and the amino acid sequence of an Fc-region.

In one or more exemplary embodiments, the present disclosure relates to a fusion-protein construct that contains a fungus binding peptide motif as disclosed herein and the amino acid sequence of an Fc-region.

In one or more exemplary embodiments, the present disclosure relates to a fusion-protein construct that contains a fungus binding motif having at least 80% sequence identity to SEQ ID NO: 2 and the amino acid sequence of an Fc-region.

In one or more exemplary embodiments, the present disclosure relates to a fusion-protein construct that contains a fungus binding motif that is at least 10 amino acids long and having at least 80% sequence identity to SEQ ID NO: 2 and the amino acid sequence of an Fc-region.

The fungus binding amino acid and/or fungus binding peptide motif as disclosed herein can be conjugated to an Fc-region from a human immunoglobulin or a non-human immunoglobulin.

In one or more exemplary embodiments, the Fc-region is from a human immunoglobulin selected from the group consisting of an IgG, IgA, IgM, IgE, and IgD.

In one or more exemplary embodiments, the human immunoglobulin Fc-region is one of the immunoglobulin isotypes selected from the list consisting of an IgG, IgA or IgD Fc-region.

In one or more exemplary embodiments, the human immunoglobulin Fc-region is one of the immunoglobulin isotypes selected from the list consisting of an IgG1, IgG2, IgG3, IgG4, IgA1, IgA2 or IgD Fc-region.

In one or more exemplary embodiments, the Fc-region is from a human immunoglobulin selected from the group consisting of an IgG and IgM.

In one or more exemplary embodiments, the Fc-region is from a human immunoglobulin selected from the group consisting of an IgG1, IgG2, IgG3, IgG4, IgA1, IgA2, IgM, IgE, and IgD.

In one or more exemplary embodiments, the Fc-region is from a human immunoglobulin selected from the group consisting of an IgG1, IgG2, IgG3, IgG4, and IgM In one or more exemplary embodiments, the Fc-region is a human IgG1.

In one or more exemplary embodiments, the Fc-region is a human IgG2.

In one or more exemplary embodiments, the Fc-region is a human IgG3.

In one or more exemplary embodiments, the Fc-region is a human IgG4.

In one or more exemplary embodiments, the Fc-region is a human IgA1.

In one or more exemplary embodiments, the Fc-region is a human IgA2.

In one or more exemplary embodiments, the Fc-region is a human IgM.

In one or more exemplary embodiments, the Fc-region is a human IgE.

In one or more exemplary embodiments, the Fc-region is a human IgD.

As described above, the fungus binding amino acid and/or fungus binding peptide motif as disclosed herein can also be conjugated to an Fc-region from a non-human immunoglobulin. Thus, in one or more exemplary embodiments, the Fc-region is a non-human Fc-region selected from the list consisting of canine, horse, bovine, sheep, swine, goat, mink, ferret, cat, rat, mouse, rabbit and guinea pig.

In one or more exemplary embodiments, the Fc-region is a non-human Fc-region selected from the list consisting of canine, horse, bovine, sheep, swine, goat, rat, mouse, rabbit and guinea pig.

In one or more exemplary embodiments, the Fc-region is a non-human Fc-region selected from the list consisting of, goat, rat, mouse, rabbit and guinea pig.

In one or more exemplary embodiments, the Fc-region is a non-human Fc-region selected from the list consisting of, goat, rabbit, and guinea pig.

In one or more exemplary embodiments, the Fc-region is a rat Fc-region.

In one or more exemplary embodiments, the Fc-region is a rat IgG Fc-region.

In one or more exemplary embodiments, the Fc-region is a mouse Fc-region.

In one or more exemplary embodiments, the Fc-region is a mouse IgG Fc-region.

In one or more exemplary embodiments, the Fc-region is a rabbit Fc-region.

In one or more exemplary embodiments, the Fc-region is a guinea pig Fc-region.

In one or more exemplary embodiments, the Fc-region is a guinea pig IgG Fc-region.

### Fusion Protein Linkers

Fusion proteins may also contain linkers that are located between the protein constituent parts of the fusion protein and thereby affect how each protein constituent part can move in relation to the other protein constituent parts. A poly-glycine linker consisting of a series of glycine residues, for example, can be used to increase mobility of the protein constituent parts, by providing a region that can curl and form an adaptable shape depending on the environment the fusion protein is currently in. Such linkers and methods for introducing these into fusion proteins are well known in the prior art.

Thus, in one or more exemplary embodiments, the present disclosure relates to a fungus binding fusion protein as disclosed herein, that further comprises a linker connecting the fungus binding amino acid or the fungus binding peptide motif to the fusion partner e.g., the exemplified Fc-region.

In one or more exemplary embodiments, the linker is a hinge-region connecting the fungus binding amino acid or the fungus binding peptide motif to the fusion partner.

### Peptibodies

A peptibody is a peptide construct comprising at least two peptide moieties, a biologically active peptide and an Fc-region, wherein the biologically active peptide is grafted onto an Fc region. This makes peptibodies both an attractive and flexible alternative to monoclonal antibodies in any process or method that traditionally makes use of monoclonal antibodies.

A peptide-coupled component, e.g., an Fc region, can improve the pharmacokinetics and increase the in vivo half-life of the compound. An Fc region can also be manipulated to further increase the effectiveness and further prolong the half-life of the construct. An Fc region can for example be designed to be recycled in the endocytic pathway in host cells in order to increase the half-life of the construct. The compound can also be pegylated to extent the half-life and bioavailability.

The Fc region can be manipulated to change the mode of interaction with components from the immune system to increase/decrease the inflammatory process by e.g., changing the interaction with C1q from the complement system.

An Fc region can benefit the production of the construct by improving the structural and biochemical stability and by easing the purification process.

The first formulation made with the peptide is a fusion protein containing the peptide, a hinge region and the Fc-region of a human IgG1. This so-called peptibody mimics the structure, function and biological half-life of natural immunoglobulins, but has the unique peptide binding region instead of the antigen-binding Fab fragment. An example of a generalized structure of a peptibody is shown in FIG. 19. The peptibody was produced recombinantly using Expi293 cells and purified using protein G sepharose.

### A Fungus Binding Peptibody

Thus, in one or more exemplary embodiments, a fungus binding peptibody is a fungus binding amino acid as disclosed herein grafted onto an Fc-region from an immunoglobulin.

In one or more other exemplary embodiments, the fungus binding peptibody comprise a fungus binding peptide motif as disclosed herein grafted onto an Fc-region from an immunoglobulin.

As described above, the Fc-region can both be a human or a non-human immunoglobulin.

In one or more other exemplary embodiments, the peptibody do not bind to a human cell.

In one or more exemplary embodiments, the fungus binding peptibody comprise a fungus binding amino acid having at least 70% sequence identity to SEQ ID NO: 1 conjugated to an Fc-region.

In one or more exemplary embodiments, the fungus binding peptibody comprise a fungus binding motif having at least 80% sequence identity to SEQ ID NO: 2 and conjugated to an Fc-region.

In one or more exemplary embodiments, the fungus binding peptibody comprise a fungus binding motif that is at least 10 amino acids long, said binding motif having at least 80% sequence identity to SEQ ID NO: 2 conjugated to an Fc-region.

In one or more exemplary embodiments, the present disclosure relates to a fungus binding peptibody, further comprising a hinge region.

In one or more exemplary embodiments, the present disclosure relates to a fungus binding peptibody, wherein the human immunoglobulin Fc-region is selected from the list consisting of an IgG1, IgG2, IgG3, IgG4, IgA1, IgA2, IgM, IgE, or IgD Fc-region.

In one or more exemplary embodiments, the present disclosure relates to a fungus binding peptibody, wherein the human immunoglobulin Fc-region is one of the immunoglobulin isotypes selected from the list consisting of an IgG, IgA, IgM, IgE or IgD Fc-region.

In one or more exemplary embodiments, the present disclosure relates to a fungus binding peptibody, wherein the human immunoglobulin Fc-region is one of the immunoglobulin isotypes selected from the list consisting of an IgG1, IgG2, IgG3, IgG4, IgA1, IgA2, IgM, IgE or IgD Fc-region.

In one or more exemplary embodiments, the present disclosure relates to a fungus binding peptibody, wherein the Fc-region is a human IgG1.

In one or more exemplary embodiments, the present disclosure relates to a fungus binding peptibody, wherein the Fc-region is a human IgG2.

In one or more exemplary embodiments, the present disclosure relates to a fungus binding peptibody, wherein the Fc-region is a human IgG3.

In one or more exemplary embodiments, the present disclosure relates to a fungus binding peptibody, wherein the Fc-region is a human IgG4.

In one or more exemplary embodiments, the present disclosure relates to a fungus binding peptibody, wherein the Fc-region is a human IgA1.

In one or more exemplary embodiments, the present disclosure relates to a fungus binding peptibody, wherein the Fc-region is a human IgA2.

In one or more exemplary embodiments, the present disclosure relates to a fungus binding peptibody, wherein the Fc-region is a human IgM.

In one or more exemplary embodiments, the present disclosure relates to a fungus binding peptibody, wherein the Fc-region is a human IgE.

In one or more exemplary embodiments, the present disclosure relates to a fungus binding peptibody, wherein the Fc-region is a human IgD.

In one or more exemplary embodiments, the present disclosure relates to a fungus binding peptibody, wherein the Fc-region is a non-human Fc-region.

In one or more exemplary embodiments, the present disclosure relates to a fungus binding peptibody, wherein the Fc-region is a non-human Fc-region is selected from the list consisting of list consisting of canine, horse, bovine, sheep, swine, goat, mink, ferret, cat, rat, mouse, rabbit and guinea pig.

In one or more exemplary embodiments, the present disclosure relates to a fungus binding peptibody, wherein the Fc-region is a non-human Fc-region is selected from the list consisting of list consisting of canine, horse, bovine, sheep, swine, goat, rat, mouse, rabbit and guinea pig.

In one or more exemplary embodiments, the present disclosure relates to a fungus binding peptibody, wherein the Fc-region is a non-human Fc-region is selected from the list consisting of list consisting of goat, rat, mouse, rabbit and guinea pig.

In one or more exemplary embodiments, the present disclosure relates to a fungus binding peptibody, wherein the Fc-region is a non-human Fc-region is selected from the list consisting of list consisting of goat, rat, and guinea pig.

In one or more exemplary embodiments, the present disclosure relates to a fungus binding peptibody, wherein the Fc-region is a rat Fc-region.

In one or more exemplary embodiments, the present disclosure relates to a fungus binding peptibody, wherein the Fc-region is a rat IgG Fc-region.

In one or more exemplary embodiments, the present disclosure relates to a fungus binding peptibody, wherein the Fc-region is a mouse Fc-region.

In one or more exemplary embodiments, the present disclosure relates to a fungus binding peptibody, wherein the Fc-region is a mouse IgG Fc-region.

In one or more exemplary embodiments, the present disclosure relates to a fungus binding peptibody, wherein the Fc-region is a rabbit Fc-region.

In one or more exemplary embodiments, the present disclosure relates to a fungus binding peptibody, wherein the Fc-region is a rabbit IgG Fc-region.

In one or more exemplary embodiments, the present disclosure relates to a fungus binding peptibody, wherein the Fc-region is a guinea pig Fc-region.

In one or more exemplary embodiments, the present disclosure relates to a fungus binding peptibody, wherein the Fc-region is a guinea pig IgG Fc-region.

### Peptibody Formulae I

In one or more exemplary embodiments, the present disclosure relates to a fungus binding peptibody having the structure defined in Formula (I);

X1-L1-F1 Formula (I):
wherein,
   X1 is a fungus binding amino acid selected from the group consisting of P1, P2 and P3, where;
      P1 is a fungus binding amino acid comprising a binding part, the binding part having at least 70% sequence identity towards SEQ ID NO: 1,
      P2 is a fungus binding motif comprising a binding motif having at least 80% sequence identity towards SEQ ID NO: 2,
      P3 is a fungus binding motif comprising a binding motif being at least 10 amino acids long and having at least 80% sequence identity to SEQ ID NO: 2
   F1 is an Fc Region, and
   L1 is a peptide bond, linker or hinge region connecting X1 to F1.

In one or more exemplary embodiments, the present disclosure relates to a fungus binding peptibody having the structure defined in Formula (II);

X1*y-L1-F1 Formula (II):
wherein,
   X1 is selected from the group consisting of P1 or P2, wherein
      P1 is a peptide according to claim 1,
      P2 is a peptide according to claim 2
   F1 is an Fc Region, and
   L1 is a peptide bond, linker or hinge region connecting X1 to F1
   y is the number of fungus binding peptides
wherein y=1-10, such as 1, 2, 3, 4, 5, 6, 7, 8, 9 or 10.

### Fc-Region

In the present context, an Fc-region is the tail region of an antibody [fragment crystallizable region (Fc region)] that interacts with cell surface receptors called Fc receptors and some proteins of the complement system. This property allows antibodies to activate the immune system.

The conjugation/covalent attachment of a peptide of interest to an Fc-region provides a number of characteristics to the peptide construct that are useful for therapeutical and diagnostic uses. For example, the Fc-region confers increased half-life in serum onto the peptibody and allows for easy separation/isolation of the peptide construct through a high affinity to Protein A and Protein G.

The addition of the Fc-region also facilitates detection of peptibody binding to a fungus in a sample or a patient by allowing for immunoblotting targeting the Fc-region using known anti-Fc antibodies.

A therapeutic advantage in the case of a human IgG Fc-region is that this Fc-region also provides direct activation of the specific immune response upon fungus binding when delivered to the bloodstream of a human patient in need thereof.

A diagnostic advantage in the case of non-human Fc-regions is that non-human Fc-regions will not cross-react with host/subject immunoglobulin Fc-regions during the detection steps.

Thus, in one or more exemplary embodiments, the present disclosure relates to a fungus binding amino acid, a fungus binding peptide motif, a fungus binding peptibody or a peptibody according to formula (I) as defined herein conjugated to an Fc-region from a human immunoglobulin, wherein the Fc-region is chosen from the list of IgG1, IgG2, IgG3, IgG4, IgA1, IgA2, IgM, IgE, or IgD Fc-region.

In one or more exemplary embodiments, the present disclosure relates to a fungus binding amino acid, a fungus binding peptide motif, a fungus binding peptibody or a peptibody according to formula (I) as defined herein conjugated to an Fc-region from a non-human immunoglobulin.

### Attachment of Bioactive Compounds

A bioactive compound is any compound or peptide that has an effect in or on an organism, tissue or cell. It is commonly known that such bioactive compounds can be attached to other carrier molecules such as for example antibodies in order to either facilitate detection of a molecular target or facilitate delivery of the bioactive compound to a molecular target. Methods for attachment of bioactive molecules onto a carrier molecule such as by conjugation is well known in the prior art.

Thus, in one or more exemplary embodiments, the present disclosure relates to fungus binding amino acids, fungus binding peptide motifs, fungus binding fusion proteins and/or fungus binding peptibodies as disclosed herein that are modified by the attachment of bioactive compounds.

In one or more exemplary embodiments, a bioactive compound is selected from a list consisting of fluorophores, fluorescent/phosphorescent proteins or compounds, radiolabeled compounds, antibiotics, enzymatically active peptides, molecular probes, functional tags such as HIS/FLAG-tags and/or PEG.

In one or more exemplary embodiments, the peptibody is conjugated to an imaging agent or a radioligand.

### Antibiotic Conjugates

In the present context, an antibiotic/antifungal conjugate, is an antibiotic/antifungal that is conjugated onto a carrier molecule. In one example of such a carrier molecule is an antibody. The antibody enables specific immunorecognition of the target organism and brings the conjugated antibiotic/antifungal into contact with the target organism, delivering the antibiotic/antifungal directly to where it is needed.

In the same way, the isolated fungus binding amino acids, isolated fungus binding peptide motifs, fungus binding fusion proteins and/or fungus binding peptibodies as disclosed herein can take on the same targeting role as the antibody and provide delivery of an antibiotic/antifungal directly to the desired target.

Thus, in one or more exemplary embodiments, the present disclosure relates to an isolated fungus binding amino acid, fungus binding peptide motif, fungus binding fusion protein and/or a fungus binding peptibody as disclosed herein onto which an antibiotic/antifungal is attached.

In one or more exemplary embodiments, the present disclosure relates to an isolated fungus binding amino acid, fungus binding peptide motif, fungus binding fusion protein and/or a fungus binding peptibody as disclosed herein that is conjugated to an antibiotic/antifungal.

In one or more exemplary embodiments the isolated fungus binding amino acids, isolated fungus binding peptide motifs, fungus binding fusion proteins and/or fungus binding peptibodies as disclosed herein are conjugated to antibiotics such as amphotericin B, triazoles and/or radionuclides.

In one or more exemplary embodiments the isolated fungus binding amino acids, isolated fungus binding peptide motifs, fungus binding fusion proteins and/or fungus binding peptibodies as disclosed herein are conjugated to cytotoxic drugs and toxic radionucleotides.

Thus, in one or more exemplary embodiments, the present disclosure relates to an isolated fungus binding amino acid sequence comprising a binding part, the binding part having at least 70% sequence identity to SEQ ID NO: 1 that is conjugated to an antibiotic/antifungal.

In one or more exemplary embodiments, the present disclosure relates to an isolated fungus binding motif comprising a binding motif having at least 80% sequence identity towards SEQ ID NO: 2 that is conjugated to an antibiotic/antifungal.

In one or more exemplary embodiments, the present disclosure relates to an isolated fungus binding motif comprising a binding motif being at least 10 amino acids long and having at least 80% sequence identity towards SEQ ID NO: 2 that is conjugated to an antibiotic/antifungal.

In one or more exemplary embodiments, the present disclosure relates to a fungus binding fusion peptide as defined herein that is conjugated to an antibiotic/antifungal.

In or more exemplary embodiments, the present disclosure relates to a fungus binding peptibody as defined herein that is conjugated to an antibiotic/antifungal.

In or more exemplary embodiments, the present disclosure relates to a fungus binding peptibody according to Formulas (I) that is conjugated to an antibiotic/antifungal.

### PEGylation

PEGylation is the process of attaching polyethylene glycol (PEG) polymers onto molecules and commonly peptides. The attachment of PEG can result in changes in the physiochemical properties of the target molecule and may include changes in for example conformation, electrostatic binding, and hydrophobicity. These changes can increase systemic retention of the target molecule, improved solubility, reduced dosage frequency requirements, reduced toxicity and enhanced protection from proteolytic degradation.

Thus, in the present context, the isolated fungus binding amino acids, isolated fungus binding peptide motifs, fungus binding fusion proteins and/or fungus binding peptibodies as disclosed herein can be chemically modified by the addition of PEG.

In one or more exemplary embodiments, the present disclosure relates to fungus binding amino acids that are PEGylated.

### Labelling or Tagging

In one or more exemplary embodiments of the present disclosure an isolated fungus binding peptide, isolated fungus binding peptide motif or fungus binding peptibody as disclosed herein are further modified in order to achieve various beneficial properties.

In one or more exemplary embodiments conjugation of the isolated fungus binding amino acid sequence, isolated fungus binding peptide motif or fungus binding peptibodies as defined herein to signal molecules further enable easy detection of fungal cells or fungal cell fragments in a sample or in vivo by monitoring the signal molecule.

Thus, in one or more exemplary embodiments the isolated fungus binding peptide, isolated fungus binding peptide motif or fungus binding peptibodies as disclosed herein are conjugated to phosphorescent molecules, fluorescent molecules such as fluorescein or fluorescent proteins, e.g. GFP, YFP, radiolabeled molecules or affinity based systems such as biotin or streptavidin that are helpful in enabling detection. Such molecules and methods are generally known in the prior art and is further exemplified by the use of biotin tagged peptides in example 3 and as shown in FIGS. 9-16.

In one or more exemplary embodiments the isolated fungus binding peptide, isolated fungus binding peptide motif or fungus binding peptibodies as disclosed herein are tagged with a molecular tag such as HIS8, HIS6, HIS4 or FLAG-tags, facilitating peptide purification and enabling antibody detection by antibody binding of the molecular tag.

### Nucleic Acid

The term nucleic acid in the present context is used to describe DNA and RNA, members of a family of biopolymers, and is synonymous with polynucleotide. Thus, the present disclosure relates to a nucleotide sequence which encodes the polypeptides described herein.

In one or more exemplary embodiments, the present disclosure relates to an isolated nucleic acid encoding a fungus binding amino acid sequence comprising a binding part, the binding part having at least 70% sequence identity to SEQ ID NO: 1.

In one or more exemplary embodiments, the present disclosure relates to an isolated nucleic acid encoding a fungus binding motif comprising a binding motif having at least 80% identity to SEQ ID NO: 2.

In one or more exemplary embodiments, the present disclosure relates to an isolated nucleic acid encoding a fungus binding motif comprising a binding motif being at least 10 amino acids long and having at least 80% sequence identity towards SEQ ID NO: 2.

### Nucleic Acid Construct

The present disclosure also relates to a nucleic acid construct comprising the polynucleotides operably linked to one or more control sequences. A nucleic acid construct within the present context is an artificial construct comprising a nucleic acid insert that is integrated into or borne by a vector. The vector can be delivered via transformation/transfection to a host cell by for example physical, chemical, or viral methods and allow the nucleic acid inserts to be replicated or expressed in the host cell. Such methods for delivering a vector into a host cell are well known to the skilled person.

In one or more exemplary embodiments, the present disclosure relates to a nucleic acid construct encoding a fusion-protein, the fusion-protein comprising a fungus binding amino acid comprising a binding part, the binding part having at least 70% sequence identity to SEQ ID NO: 1 and the amino acid sequence of an Fc-region from a human immunoglobulin.

In one or more exemplary embodiments, the present disclosure relates to a nucleic acid construct encoding a fusion-protein, the fusion-protein containing a fungus binding motif comprising a binding motif having at least 80% identity to SEQ ID NO: 2 and the amino acid sequence of an Fc-region from a human immunoglobulin.

In one or more exemplary embodiments, the present disclosure relates to nucleic acid constructs or vectors that facilitate transformation into a suitable host cell.

In one or more exemplary embodiments, the nucleic acid constructs or vectors of the present disclosure comprise one or more control elements (expression start/stop/transposable elements).

In one or more exemplary embodiments, the present disclosure relates to nucleic acid constructs and vectors of use in producing fungus binding amino acid sequences, fungus binding peptide motifs, fungal binding fusion proteins or fungal binding peptibodies as disclosed herein.

### Fungal Binding Amino Acid Construct

In one or more exemplary embodiments, the present disclosure relates to a nucleic acid construct that encodes a fungus binding amino acid as disclosed herein.

In one or more exemplary embodiments, the present disclosure relates to a nucleic acid construct that encodes a fungus binding amino acid comprising a binding part, the binding part having at least 70% sequence identity to SEQ ID NO: 1.

### Fungal Binding Peptide Motif Construct

In one or more exemplary embodiments, the present disclosure relates to a nucleic acid construct that encodes a fungus binding peptide motif as disclosed herein.

In one or more exemplary embodiments, the present disclosure relates to a nucleic acid construct that encodes a fungus binding peptide motif comprising a binding motif having at least 80% sequence identity to SEQ ID NO: 2.

In one or more exemplary embodiments, the present disclosure relates to a nucleic acid construct that encodes a fungus binding motif that is at least 5 amino acids, at least 10 amino acids, at least 15 amino acids, at least 20 amino acids, at least 25 amino acids, at least 30 amino acids long.

In one or more exemplary embodiments, the present disclosure relates to a nucleic acid construct that encodes a fungus binding peptide motif comprising a binding motif that is at least 10 amino acids long, said binding motif having at least 80% sequence identity to SEQ ID NO: 2.

In one or more exemplary embodiments, the present disclosure relates to a nucleic acid construct that encodes a fungus binding motif that is at least 5 amino acids, at least 10 amino acids, at least 15 amino acids, at least 20 amino acids, at least 25 amino acids, at least 30 amino acids long, wherein said fungus binding peptide further comprises a section consisting of 5 consecutive amino acids, the 5 consecutive amino acids being the amino acid sequence KLMAR [SEQ ID NO: 2].

In one or more exemplary embodiments, the present disclosure relates to a nucleic acid construct that encodes a fungus binding motif that is at least 5 amino acids, at least 10 amino acids, at least 15 amino acids, at least 20 amino acids, at least 25 amino acids, at least 30 amino acids long, wherein said fungus binding peptide further includes a section consisting of 5 consecutive amino acids, the 5 consecutive amino acids having 80% sequence identity to the amino acid sequence KLMAR [SEQ ID NO: 2].

In one or more exemplary embodiments, the present disclosure relates to a nucleic acid construct that encodes a fungus binding motif that is at least 5 amino acids, at least 10 amino acids, at least 15 amino acids, at least 20 amino acids, at least 25 amino acids, at least 30 amino acids long, wherein said fungus binding peptide further includes a section consisting of 5 consecutive amino acids, the 5 consecutive amino acids having 60% sequence identity to the amino acid sequence KLMAR [SEQ ID NO: 2].

### Fungal Binding Fusion Protein Construct

In one or more exemplary embodiments, the present disclosure relates to a nucleic acid construct that encodes a fungus binding fusion protein as disclosed herein.

In one or more exemplary embodiments, the present disclosure relates to a nucleic acid construct that encodes a fungus binding fusion protein including an Fc-region and a fungus binding amino acid comprising a binding part, the binding part having at least 70% sequence identity to SEQ ID NO: 1 and an Fc-region.

In one or more exemplary embodiments, the present disclosure relates to a nucleic acid construct that encodes a fungus binding fusion protein comprising an Fc-region and a fungus binding peptide motif having at least 80% sequence identity to SEQ ID NO: 2.

In one or more exemplary embodiments, the present disclosure relates to a nucleic acid construct that encodes a fungus binding fusion protein comprising and Fc-region and a fungus binding peptide motif that is at least 10 amino acids long, said binding motif having at least 80% sequence identity to SEQ ID NO: 2.

### Fungal Binding Peptibody Construct

In one or more exemplary embodiments, the present disclosure relates to a nucleic acid construct that encodes a fungus binding peptibody as disclosed herein.

In one or more exemplary embodiments, the present disclosure relates to a nucleic acid construct that encodes a fungus binding peptibody including a fungus binding amino acid comprising a binding part, the binding part having at least 70% sequence identity to SEQ ID NO: 1.

In one or more exemplary embodiments, the present disclosure relates to a nucleic acid construct that encodes fungus binding peptibody comprising a fungus binding motif having at least 80% sequence identity to SEQ ID NO: 2.

In one or more exemplary embodiments, the present disclosure relates to a nucleic acid construct that encodes a fungus binding peptibody comprising a fungus binding motif that is at least 10 amino acids long, said binding motif having at least 80% sequence identity to SEQ ID NO: 2.

### Host Cells

To manufacture biotechnology products, cells can be engineered for the production of recombinant proteins including for example peptibodies. Within the context of the present disclosure a host cell is a cell that is used for harboring and expressing a nucleic acid or nucleic acid construct as defined herein. Methods for introducing nucleic acid and nucleic acid constructs into cells to provide host cells are well known in the prior art.

Thus, in one or more exemplary embodiments, the present disclosure relates to a host cell comprising a nucleic acid or nucleic acid construct encoding a fungus binding amino acid sequence according to any one of the fungi binding amino acids, fungi binding motifs, fungi binding fusion-proteins, fungi binding peptibodies and/or peptibodies of Formula (I) as disclosed herein.

In one or more exemplary embodiments, the host cell expresses a fungus binding amino acid sequence encoded by the nucleic acid or nucleic acid construct as disclosed herein.

In one or more exemplary embodiments, the present disclosure relates to a host cell that is selected from a list comprising yeast cells, bacterial cells, mammalian cells and plant cells.

In one or more exemplary embodiments, the host cell is a yeast cell.

In one or more exemplary embodiments, the host cell is a bacterial cell.

In one or more exemplary embodiments, the host cell is an E. coli bacterial cell.

In one or more exemplary embodiments, the host cell is a mammalian cell.

In one or more exemplary embodiments, the host cell is a plant cell.

### Method for Polypeptide Production

In the present context, a method for polypeptide production relates to a method wherein a host cell is cultured/cultivated to produce a polypeptide by expression of a nucleic acid or nucleic acid construct. Such methods for production of polypeptide in a host cell are well known in the prior art.

Thus, in one or more exemplary embodiment, the present disclosure relates to a method for producing a fungus binding amino acid sequence encoding fungus binding amino acids, fungus binding motifs, fungus binding fusion-proteins, fungus binding peptibodies and/or peptibodies of Formula (I) as disclosed herein, the method comprising:
culturing a host cell as defined herein, and
recovering the fungus binding amino acid sequence.

In one or more exemplary embodiments, the present disclosure relates to a method for protein production by culturing a host cell comprising a nucleic acid or nucleic acid construct as defined herein.

### Compositions

In one or more exemplary embodiments, the present disclosure relates to compositions comprising a fungus binding amino acid, a fungus binding motif, a fungus binding fusion-protein, a fungus binding peptibody and/or peptibodies of formula (I) as disclosed herein.

In one or more exemplary embodiments, the composition can be used as an antimicrobial agent.

In one or more exemplary embodiments, the composition may be the fungus binding amino acid sequences per se.

Using a Fc fusion technology, one can easily couple cytotoxic drugs and toxic radionucleotides to the constructs allowing this to be a novel antibiotic/antifungal without the need for endogenous immune assistance. This will be a significant advantage in neutropenic patients, who are usually prone to aspergillus infections.

### Pharmaceutical Compositions

In one or more exemplary embodiments, the present disclosure relates to pharmaceutical compositions having as an active ingredient a fungus binding amino acid sequence, a fungus binding motif, a fungus binding fusion-protein or a fungus binding peptibody as disclosed herein.

Pharmaceutical composition comprises in addition to the active ingredient, therapeutically inactive ingredients, such as a pharmaceutically acceptable or physiologically acceptable excipient, carrier and/or adjuvants, which are well-known to the person skilled in the art and may include, but are not limited to, solvents, emulsifiers, wetting agents, plasticizers, solubilizers (e.g. solubility enhancing agents) coloring substances, fillers, preservatives, anti-oxidants, anti-microbial agents, viscosity adjusting agents, buffering agents, pH adjusting agents, isotonicity adjusting agents, mucoadhesive substances, and the like. Examples of formulation strategies are well-known to the person skilled in the art.

In the present context a pharmaceutical composition is a mixture of ingredients suitable for administering to a subject that includes an active ingredient.

### Medical Use

The fungus binding amino acid could also be used to treat infections with bacteria (e.g., mycobacteria), other fungi, viruses (e.g., herpes viruses) or parasites (e.g., malaria parasites) that may bind the peptide sequence.

One medical composition developed is the fusion-protein construct that contains a fungus binding amino acid and the Fc-region of a human IgG. There are different reasons to include an IgG Fc region;
a) to improve the structural and biochemical stability and increase the PK/in vivo half-life of the compound,
b) to use the fusion-protein as a carrier of anti-fungal reagents,
c) to mediate fungal killing by the immune system through phagocytosis and antibody-dependent cellular cytotoxicity as an additive effect to the target sequence,
d) to activate the complement system via the classical pathway mediating further phagocytosis and complement mediated cytolysis, and
e) to have a straightforward purification method as protein A and protein G binds strongly and selectively to IgG Fc regions.

The fusion protein can be made with different formulations where the ratio between peptides and Fc regions can be changed.

The constructs can also be made with different Fc subclasses (IgG1, IgG2, IgG3, IgG4) as the subclasses have different biological properties regarding Fc-receptor interactions and complement activation abilities.

Thus, the identification of the fungi binding amino acid sequences as disclosed herein opens many possibilities for new approaches for medical use of these sequences, such as but not limited to treatment of fungal infections. The fungi binding amino acids are useful in treatment of infections caused by fungal pathogens as exemplified in example 6.

The medical use within the present context relates to the use of alleviating symptoms of a disease condition or disorder or in the treatment of a disease, condition, or disorder.

Thus, in one or more exemplary embodiments, the present disclosure relates to a composition comprising a fungus binding amino acid sequence, a fungus binding motif, a fungus binding fusion-protein, a fungus binding peptibody and/or a peptibody of formula (I) as disclosed herein for use as a medicament, use in therapy and/or prophylaxis.

Using a Fc fusion technology, one can easily couple cytotoxic drugs and toxic radionucleotides to the constructs allowing this to be a novel antibiotic/antifungal without the need for endogenous immune assistance. This will be a significant advantage in neutropenic patients, who are usually prone to aspergillus infections.

In one or more exemplary embodiments, the composition as described above is coupled to a radionucleotide.

In one or more exemplary embodiments, the composition as described above is coupled to a cytotoxic drug.

In one or more presently preferred exemplary embodiments, the composition described above is use in the treatment of an immunocompromised individual.

In one or more presently preferred exemplary embodiments, the composition described above is use in the treatment of an opportunistic fungal infection.

The first formulation made is a so-called peptibody, where two peptides substitute the Fab region of a natural IgG1, which is exemplified in FIG. 19). This peptibody was produced recombinantly using Expi293 cells. This peptibody binds to A. fumigatus as shown in FIGS. 20-22.

An in vivo mouse study discloses the therapeutic effect of the peptibody. C57BI/6J B6 mice were continuously immunosuppressed cyclophosphamide to mimic the human immunocompromised situation. The mice were then intranasally infected with 1×10⁷ A. fumigatus conidia and treated intranasally with the peptibody. The mice were divided in groups of 6 to test different dosages of peptibody; P1) PBS, P2) 0.6 mg/kg peptibody, P3) 6 mg/kg peptibody and P4) 60 mg/kg peptibody. In the PBS group, all mice died within 8 days, whereas all mice survived the study period of 2 weeks with the highest peptibody dose (FIG. 23). Moreover, there was a dose-dependent effect of the peptibody on survival (FIG. 23). Accordingly, the fungal burden in the lungs was shown to be lowered/eliminated in the peptibody treated groups (FIGS. 24 and 25).

Medical Conditions Associated with Fungal

In the present context, a medical condition which is associated with a potential fungal infection can be Aspergillosis, Aspergilloma, Blastomycosis, Bone Marrow Transplantation, Candida Urinary Tract Infection, Candidemia, Chromomycosis, Chronic Mucocutaneous Candidiasis, Coccidioidomycosis, Coccidioidomycosis, Meningitis, Cryptococcal Meningitis, Immunocompetent Host, Cryptococcal Meningitis, Immunosuppressed Host, Cryptococcosis, Cutaneous Fungal Infection, Dermatophytosis, Esophageal Candidiasis, Eumycetoma, Febrile Neutropenia, Fungal Infection Prevention, Fungal Infection Prophylaxis, Fungal Infection, Fungal Meningitis, Fungal Peritonitis, Fungal Pneumonia, Fusariosis, Histoplasmosis, Microsporidiosis, Mucormycosis, Ocular Fungal Infection, Onychomycosis, Oral Thrush, Paracoccidioidomycosis, Pseudoallescheriosis, Sporotrichosis, Systemic Candidiasis, Systemic Fungal Infection, Tinea Capitis. Tinea Versicolor, and Vaginal Yeast Infection.

In one or more presently preferred exemplary embodiments, a medical condition which is associated with a potential fungal infection is pulmonary aspergillosis, cerebral aspergillosis, invasive aspergillosis, systemic aspergillosis, disseminated aspergillosis or aspergilloma.

In one or more presently preferred exemplary embodiments, a medical condition which is associated with a potential fungal infection is aspergilloma.

In one or more presently preferred exemplary embodiments, a medical condition which is associated with a potential fungal infection is disseminated aspergillosis.

In one or more presently preferred exemplary embodiments, a medical condition which is associated with a potential fungal infection is systemic aspergillosis.

In one or more presently preferred exemplary embodiments, a medical condition which is associated with a potential fungal infection is pulmonary aspergillosis.

In one or more presently preferred exemplary embodiments, a medical condition which is associated with a potential fungal infection is cerebral aspergillosis.

In one or more presently preferred exemplary embodiments, a medical condition which is associated with a potential fungal infection is invasive aspergillosis.

### Drug Resistant Strains

Certain strains of fungi have become more resistant to antifungal medicines. They're known as superbugs. These fungi continue to multiply and cause infections even when you take medication, such as the current three major classes of antifungal medicines: azoles, echinocandin and polyenes. The emergence of multidrug-resistant (MDR) fungal strains of Candida auris and azole-resistant Aspergillus fumigatus were highlighted in a recent CDC report: Antibiotic Resistance Threats in the United States. Thus, conventional antifungals used to treat fungal infections are no longer as effective, leading to increased mortality.

Thus, in one or more exemplary embodiments, the composition comprising a fungus binding amino acid sequence as described herein is particularly useful for the treatment of drug-resistant fungal strains.

### Combination Treatment

The combination of two or more antifungal drugs with different modes of action in combination is recommended. The concept of combination therapy is based on the synergistic or additive potential of two or more drugs, to improve therapeutic efficacy. It also delays the development of the resistance form of the fungi.

Thus, in one or more exemplary embodiments, the composition comprising a fungus binding amino acid sequence as described herein is particularly useful for the treatment of drug-resistant fungal strains in combination with other antifungal drugs with different modes of action, such as but not limited to amphotericin B, flucytosine, and fluconazole, alone or in combination.

### Polyene Antifungal Agent

Polyene antimycotics, sometimes referred to as polyene antibiotics, are a class of antimicrobial polyene compounds that target fungi. Amphotericin B, nystatin, and natamycin are examples of polyene antimycotics. They are a subgroup of macrolides.

Thus, in one or more exemplary embodiments, the composition comprising a fungus binding amino acid sequence as described herein is particularly useful for the treatment of drug-resistant fungal strains in combination with a polyene antifungal agent.

### Azoles Antifungal Agent

Azoles function by disrupting ergosterol biosynthesis through inhibition of the cytochrome P-450-dependent enzyme lanosterol 14-α-demethylase. Azole antifungal agents have added greatly to the therapeutic options for treatment of systemic fungal infections. The azoles that are available for systemic use can be classified into two groups: the triazoles (fluconazole, itraconazole, voriconazole, posaconazole, and isavuconazole) and the imidazoles (ketoconazole).

Thus, in one or more exemplary embodiments, the composition comprising a fungus binding amino acid sequence as described herein is particularly useful for the treatment of drug-resistant fungal strains in combination with an azole antifungal agent.

### Echinocandin Antifungal Agent

Echinocandins are a class of antifungal drugs that inhibit the synthesis of β-glucan in the fungal cell wall via noncompetitive inhibition of the enzyme 1,3-β glucan synthase. The class has been termed the “penicillin of antifungals,” along with the related papulacandins, as their mechanism of action resembles that of penicillin in bacteria. β-glucans are carbohydrate polymers that are cross-linked with other fungal cell wall components, the fungal equivalent to bacterial peptidoglycan. Caspofungin, micafungin, and anidulafungin are semisynthetic echinocandin derivatives with limited clinical use due to their solubility, antifungal spectrum, and pharmacokinetic properties.[5]. They are used to treat invasive fungal infections and show good activity against amphotericin B-resistant and fluconazole-resistant Candida guilliermondii. Caspofungin was the first drug in this class to be approved.

Thus, in one or more exemplary embodiments, the composition comprising a fungus binding amino acid sequence as described herein is particularly useful for the treatment of drug-resistant fungal strains in combination with an echinocandin antifungal agent.

### Papulacandins

Papulacandins are antibiotics, particularly active against Candida albicans and several other yeasts, and was originally isolated from a strain of Papularia sphaerosperma. The fermentation, isolation, physico-chemical properties and biological activity of the five structurally related papulacandins A, B, C, D and E are reported. Papulacandin B, the main component, was assigned the formula of C47H64O17.

Thus, in one or more exemplary embodiments, the composition comprising a fungus binding amino acid sequence as described herein is particularly useful for the treatment of drug-resistant fungal strains in combination with a papulacandin antifungal agent.

Thus, in one or more presently preferred exemplary embodiments, the composition comprising a fungus binding amino acid sequence as described herein is particularly useful for the treatment of drug-resistant fungal strains in combination with Papulacandin B.

### Delivery

Drug delivery refers to approaches, formulations, manufacturing techniques, storage systems, and technologies involved in transporting a pharmaceutical compound to its target site to achieve a desired therapeutic effect. Principles related to drug preparation, route of administration, site-specific targeting, metabolism, and toxicity are used to optimize efficacy and safety, and to improve patient convenience and compliance. Throughout history, oral administration has been regarded as the most convenient mode of drug delivery, as it requires minimal expertise and invasiveness. Although oral delivery works well for small-molecule drugs, oral delivery of macromolecules (particularly proteins and peptides) has been limited by acidic conditions in the stomach and low permeability across the intestinal epithelium. Accordingly, the large numbers of biologic drugs that have become available may require administration by injection or infusion.

In one in one or more exemplary embodiments, the composition comprising a fungus binding amino acid sequence as described herein is delivered by nasal injection/delivery, subcutaneous injection/delivery, intravenous injection/delivery, inhalation or intratracheal injection/delivery, transdermal delivery, and/or oral delivery.

### Nasal Injection/Delivery

In some situations, the nasal delivery route is preferred for systemic therapy because it provides an agreeable alternative to injection or pills. Substances can be assimilated extremely quickly and directly through the nose. Many pharmaceutical drugs exist as nasal sprays for systemic administration (e.g. sedative-analgesics, treatments for migraine, osteoporosis and nausea). Other applications include hormone replacement therapy, treatment of Alzheimer's disease and Parkinson's disease.

Nasal sprays are seen as a more efficient way of transporting drugs with potential use in crossing the blood-brain barrier.

In one in one or more exemplary embodiments, the composition comprising a fungus binding amino acid sequence as described herein is delivered by nasal injection/delivery.

### Subcutaneous Injection/Delivery

A subcutaneous injection is administered as a bolus into the subcutis, the layer of skin directly below the dermis and epidermis, collectively referred to as the cutis. The instruments are usually a hypodermic needle and a syringe. Subcutaneous injections are highly effective in administering medications such as insulin, morphine, diacetylmorphine and goserelin. Subcutaneous administration may be abbreviated as SC, SQ, subcu, sub-Q, SubQ, or subcut. Subcut is the preferred abbreviation to reduce the risk of misunderstanding and potential errors.

Subcutaneous tissue has few blood vessels and so drugs injected here are for slow, sustained rates of absorption, often with some amount of depot effect. Compared with other routes of administration, it is slower than intramuscular injections but still faster than intradermal injections. Subcutaneous infusion (as opposed to subcutaneous injection) is similar but involves a continuous drip from a bag and line, as opposed to injection with a syringe.

In one in one or more exemplary embodiments, the composition comprising a fungus binding amino acid sequence as described herein is delivered by subcutaneous injection/delivery.

### Intravenous Injection/Delivery

Intravenous therapy (abbreviated as IV therapy) is a medical technique that administers fluids, medications and nutrients directly into a person's vein. It may also be used to administer medications or other medical therapy.

The intravenous route is the fastest way to deliver medications and fluid replacement throughout the body as they are introduced directly into the circulatory system and thus quickly distributed.

Many therapies are administered as a “bolus” or one-time dose, but they may also be administered as an extended infusion or drip. The act of administering a therapy intravenously, or placing an intravenous line (“IV line”) for later use, is a procedure which should only be performed by a skilled professional.

In one in one or more exemplary embodiments, the composition comprising a fungus binding amino acid sequence as described herein is delivered by intravenous injection/delivery.

### Inhalation or Intratracheal Injection

The use of the lungs for the systemic delivery of medicines is a very efficient tool. Inhaled insulin for diabetics made the way and, so far, the only available inhaled protein-based drug for systemic effect.

The high surface area and high permeability of the lungs make them an ideal site for rapid systemic delivery of macromolecules and small-molecule drugs. Small molecules are absorbed more rapidly through the lungs than through the gastrointestinal tract, with higher bioavailabilites and reduced first-pass metabolism by enzymes. The lungs are significantly permeable to many peptides and proteins, with the rate of absorption decreasing with increasing molecular mass.

In one in one or more exemplary embodiments, the composition comprising a fungus binding amino acid sequence as described herein is delivered by inhalation or intratracheal injection/delivery.

### Oral Delivery

Oral drug delivery is the most preferred route of drug delivery of pharmaceuticals encompassing number of diseases which have been successfully treated. Owing to its potential advantages including well-established delivery system, patient friendly, convenient, cost effective, and noninvasiveness, it has been the most favored drug delivery system in pharmaceutical field.

In one in one or more exemplary embodiments, the composition comprising a fungus binding amino acid sequence as described herein is delivered by oral delivery.

### Transdermal Delivery

Transdermal delivery is to apply the drugs or medicines to the skin. Transdermal delivery systems have become a successful alternative for a continuous drug delivery on demand.

In the present context, transdermal delivery relates to the application of composition disclosed herein onto the epidermis.

In one or more exemplary embodiments, the composition penetrates through the epidermis and dermis and enters circulation in the body.

In one or more exemplary embodiments, nanoparticles may be used for delivery of the compositions.

Several formulations have been used for transdermal delivery and they include, but are not limited to nanoemulsions, dendrimers, lipid nanoparticles, polymeric nanoparticles carbon nanotubes and vesicular systems such as liposomes, niosomes, transfersomes, ethosomes.

Thus, in one or more exemplary embodiments, the compositions can be formulated for transdermal delivery using nanoemulsions, dendrimers, lipid nanoparticles, polymeric nanoparticles carbon nanotubes, vesicles, liposomes, niosomes, transfersomes and ethosomes.

In one or more exemplary embodiments, the compositions are formulated for transdermal delivery using nanoparticles.

In one or more exemplary embodiments, the compositions are formulated for transdermal delivery using nanoemulsions.

In one or more exemplary embodiments, the compositions are formulated for transdermal delivery using liposomes.

In one or more exemplary embodiments, the compositions are formulated for transdermal delivery using lipid nanoparticles.

In one or more exemplary embodiments, the transdermal formulations disclosed above is delivered onto the epidermis using for example a patch, drug reservoir, gel, ointment, or cream.

In one or more exemplary embodiments, the transdermal formulations disclosed above is delivered onto the epidermis using a patch.

In one or more exemplary embodiments, the transdermal formulations disclosed above is delivered onto the epidermis using a gel.

In one or more exemplary embodiments, the transdermal formulations disclosed above is delivered onto the epidermis using a cream.

### Dosage Regimens

In one in one or more exemplary embodiments, the present disclosure relates to a dosage regime comprising delivery of a composition comprising a fungus binding amino acid sequence as described herein comprising 3 deliveries at T1=1 hour, T2=8 hours, and T3=24 hours

### Diagnostic Use

The identification of the fungus binding amino acid sequence opens many possibilities for new approaches to detection and thus diagnostic identification of fungal infections. The fungus binding amino acid sequence could also be used for diagnosis of invasive fungal infections. In vitro detection can be done via different reagents conjugated to the fungus binding amino acid sequence e.g. biotin-tag, fluorescent tag or enzymes directly coupled to the peptide construct. In vivo detection can be done via radiolabeling the peptide and subsequent diagnostic imaging.

The fungus binding amino acid sequence could be used as a diagnostic tool to detect whether the patient has a fungal infection using plasma, lung fluid, spinal fluid or by in vivo imaging. Various detection systems could be coupled to the peptide e.g., a biotin-tag or fluorophore. The peptide could also be coupled to an Fc region which allows the peptide binding to be detected via the Fc region, which is the backbone in many well-established test systems. An Fc region or other stabilizing components could also work as a scaffold for radiolabeling or for carrying other detection reagents such as fluorophores.

The fungus binding amino acid sequence could also be used to diagnose and follow infections with bacteria (eg mycobacteria), other fungi, viruses (e.g., herpes viruses) or parasites (e.g., malaria parasites) that bind the peptide sequence.

In the present context, a diagnosis is the process of determining which disease or condition explains a subject's symptoms and signs. An integrated part of this process relates to the collection of data and information about a symptoms or signs of disease in a subject as well as the identification via the fungus binding amino acid sequence. Diagnostic use in the present context therefore relates to the use of compounds, compositions, or equipment in the collection of medical data with regards to a subject that will assist the medical practitioner in establishing a clinical picture and a diagnosis.

Thus, in one or more exemplary embodiments, the present disclosure relates to a composition comprising a fungus binding amino acid sequence according to any one of the herein described fungus binding amino acids, fungus binding motifs, the fungus binding fusion-proteins, the fungus binding peptibodies and/or peptibodies of formula (I) for use in diagnosis of a fungal infection. The fungal infection may be an opportunistic fungal infection. Opportunistic fungi are defined above.

As described above, the isolated fungus binding peptides of the disclosure are particularly useful in detection of fungal infections caused by fungi belonging to the Aspergillus genus or the Mucorales order.

In one or more exemplary embodiments, the present disclosure relates to a method for detecting a fungus in a sample comprising identifying the binding of the fungus binding amino acid sequence as disclosed herein to a fungus or fungal fragments within the sample.

In one or more exemplary embodiments, the present disclosure relates to a method for detecting a fungus in a sample, where binding of the fungus binding amino acid sequence as disclosed herein to the sample can be identified by immunodetection or by measuring a signal from a radionuclide or a signaling molecule in the sample.

### Detection Method

In one or more exemplary embodiments, the method of detection is based on immunodetection.

In one or more exemplary embodiments the immunodetection is performed using a primary antibody targeting either the fungus binding amino acid sequence, or a molecular tag, or Fc region attached to the fungus binding amino acid.

The primary antibody can then be targeted by a secondary antibody tagged with a dye.

In one or more exemplary embodiments, the method of detection is dye staining, in particular fluorescein isothiocyanate (FITC) staining as disclosed or other fluorophores exited by a blue laser e.g. R-phycoerythrin (PE). Also, fluorophores exited by a red laser e.g. Allophycocyanin (APC) and by a violet laser e.g. Pacific Orange.

Size separation-based methods that utilizes methods such as, but not limited to centrifugation or membrane separation techniques to separate a cellular fraction from a sample that has been treated with fungus binding amino acid sequences as disclosed herein, followed by washing, resuspension and detection of signals directly emitted by the fungus binding amino acids or by immunodetection.

### Sample

Fungi are found everywhere in and on the human body. Thus, all types of samples obtainable from the human body can in principle by examined for the presence of fungus. Skin, hair and nail tissue are collected for microscopy and culture (mycology) to establish or confirm the diagnosis of a fungal infection.

Body fluids, bodily fluids, or biofluids are liquids within the human body and are often easy assessable. Thus, in one or more exemplary embodiments, the sample is a body fluid.

In the present context, a specific body fluid can be selected from the group consisting of blood, saliva, mucus, sputum, serum, plasma, bile, pus, urine, semen, breast milk, transudate, rheum, cerebrospinal and interstitial fluid.

In one or more exemplary embodiments, the sample is a blood sample.

In one or more exemplary embodiments, the sample is a lung fluid sample.

Bronchoalveolar lavage is a diagnostic method of the lower respiratory system in which a bronchoscope is passed through the mouth or nose into an appropriate airway in the lungs, with a measured amount of fluid introduced and then collected for examination.

In one or more exemplary embodiments, the sample is bronchoalveolar fluid sample.

In one or more exemplary embodiments, the sample is a brain fluid sample.

In one or more exemplary embodiments, the sample is a spinal fluid sample.

### Methods for Delivery to Target Physiology

Intranasal delivery of droplets comprising/containing peptibody in PBS

### Identification of Fungus Binding

The person skilled in the art can easily identify whether an amino acid sequence can be characterised as fungus binding by e.g., making an immunoblotting and dye staining (in particular fluorescein isothiocyanate (FITC) staining), as shown in the Examples.

In general, for any given peptide, it's binding activity against a fungus can be assessed in a number of standard methods, such as, but not limited to fluorescence microscopy, immunodetection, flow cytometry or other methods based on measuring a signal from a sample. This signal can either come from a tag, label or fusion added to the peptide or from a signal from a primary or secondary antibody that is tagged or labeled.

For non-microscopy methods, the signal intensity of a sample comprising a peptide of interest can be compared to e.g. a negative control sample (a peptide known to not bind a fungus) and a positive control sample (a peptide known to bind a fungus). Thus, if a given signal from a sample is stronger than the negative control, then the peptide has fungus binding activity.

This comparison will make it possible identify whether a given peptide is capable of binding a fungus and with what relative intensity (see e.g., example 3 and 5; FIGS. 9-18 and 20-22).

For microscopy methods, the detection of a signal can be compared directly to the brightfield view of the sample. Thus, if signal can be detected from the sample it can be directly mapped, whether the signal originated from the fungus or not. If the shape or localization of the signal corresponds directly to the shape or localization of the fungus in the brightfield view, then the peptide has fungus binding activity (see e.g. FIGS. 1-8). This case can be distinguished from unspecific binding, wherein binding is scattered across the sample and does not specifically co-locate with the fungus as seen in the brightfield view. Again, negative and positive controls can be utilized to further distinguish whether a peptide binds to a fungus or not by comparison.

In one or more exemplary embodiments of the present disclosure, an increase in signal intensity in a sample by at least 10%, 20%, 30%, 40%, 50%, 60%, 70%, 80%, 90% or at least 100% when compared to a negative control is indicative of a fungus binding peptide sequence binding to a fungus.

Thus, virtually any method that utilizes a tag, dye, or signal that enables detection of a signal in a sample can be used to detect if a given peptide has fungus binding activity.

### General

It should be understood that any feature and/or aspect discussed above in connection with fungus binding amino acid sequences, fungus binding peptide motifs, fungal binding fusion proteins, or fungal binding peptibodies according to the present disclosure apply by analogy to the methods described herein.

It should be understood that any feature and/or aspect discussed above in connection with fungus binding amino acid sequences, fungus binding peptide motifs, fungal binding fusion proteins, or fungal binding peptibodies according to the present disclosure apply by analogy to each other.

The terms peptide and fungus binding amino acid sequence are used interchangeably.

The following figures and examples are provided below to illustrate the present invention. They are intended to be illustrative and are not to be construed as limiting in any way.

### BRIEF DESCRIPTION OF THE FIGURES

FIG. 1 shows both a brightfield image and a fluorescence image of Aspergillus fumigatus in the resting conidia growth stage incubated together with rMASP-1. A monoclonal antibody that recognizes a MASP-1 epitope was applied and binding of rMASP-1 was assessed with fluorescence microscopy using an Alexa Fluor 488-conjugated secondary antibody.

FIG. 2 shows both a brightfield image and a fluorescence image of Aspergillus fumigatus in the resting conidia growth stage incubated without rMASP-1. A monoclonal antibody that recognizes a MASP-1 epitope was applied and binding of rMASP-1 was assessed with fluorescence microscopy using an Alexa Fluor 488-conjugated secondary antibody.

FIG. 3 shows both a brightfield image and a fluorescence image of Aspergillus fumigatus in the swollen conidia growth stage incubated together with rMASP-1. A monoclonal antibody that recognizes a MASP-1 epitope was applied and binding of rMASP-1 was assessed with fluorescence microscopy using an Alexa Fluor 488-conjugated secondary antibody.

FIG. 4 shows both a brightfield image and a fluorescence image of Aspergillus fumigatus in the swollen conidia growth stage incubated without rMASP-1. A monoclonal antibody that recognizes a MASP-1 epitope was applied and binding of rMASP-1 was assessed with fluorescence microscopy using an Alexa Fluor 488-conjugated secondary antibody.

FIG. 5 shows both a brightfield image and a fluorescence image of Aspergillus fumigatus in the germ tube growth stage incubated together with rMASP-1. A monoclonal antibody that recognizes a MASP-1 epitope was applied and binding of rMASP-1 was assessed with fluorescence microscopy using an Alexa Fluor 488-conjugated secondary antibody.

FIG. 6 shows both a brightfield image and a fluorescence image of Aspergillus fumigatus in the germ tube growth stage incubated without rMASP-1. A monoclonal antibody that recognizes a MASP-1 epitope was applied and binding of rMASP-1 was assessed with fluorescence microscopy using an Alexa Fluor 488-conjugated secondary antibody.

FIG. 7 shows both a brightfield image and a fluorescence image of Aspergillus fumigatus in the hyphae growth stage incubated together with rMASP-1. A monoclonal antibody that recognizes a MASP-1 epitope was applied and binding of rMASP-1 was assessed with fluorescence microscopy using an Alexa Fluor 488-conjugated secondary antibody.

FIG. 8 shows both a brightfield image and a fluorescence image of Aspergillus fumigatus in the hyphae growth stage incubated without rMASP-1. A monoclonal antibody that recognizes a MASP-1 epitope was applied and binding of rMASP-1 was assessed with fluorescence microscopy using an Alexa Fluor 488-conjugated secondary antibody.

FIG. 9 shows 1×10⁷ conidia/ml of heat-inactivated conidia from Aspergillus fumigatus 293 incubated with a 30 amino acid MASP-1 derived peptide tagged with a biotin tag compared to a control comprising no peptide. The biotin tag was detected with a FITC-conjugated streptavidin using flow cytometry.

FIG. 10 shows 1×10⁷ conidia/ml of heat-inactivated conidia from Aspergillus niger incubated with a 30 amino acid MASP-1 derived peptide tagged with a biotin tag compared to a control comprising no peptide. The biotin tag was detected with a FITC-conjugated streptavidin using flow cytometry.

FIG. 11 shows 1×10⁷ conidia/ml of heat-inactivated conidia from Aspergillus terreus incubated with a 30 amino acid MASP-1 derived peptide tagged with a biotin tag compared to a control comprising no peptide. The biotin tag was detected with a FITC-conjugated streptavidin using flow cytometry.

FIG. 12 shows 1×10⁷ conidia/ml of heat-inactivated conidia from Aspergillus flavus incubated with a 30 amino acid MASP-1 derived peptide tagged with a biotin tag compared to a control comprising no peptide. The biotin tag was detected with a FITC-conjugated streptavidin using flow cytometry.

FIG. 13 shows 1×10⁷ conidia/ml of heat-inactivated conidia from Lichtheimia corymbifera incubated with a 30 amino acid MASP-1 derived peptide tagged with a biotin tag compared to a control comprising no peptide. The biotin tag was detected with a FITC-conjugated streptavidin using flow cytometry.

FIG. 14 shows 1×10⁷ conidia/ml of heat-inactivated conidia from Mucor circinelloides incubated with a 30 amino acid MASP-1 derived peptide tagged with a biotin tag compared to a control comprising no peptide. The biotin tag was detected with a FITC-conjugated streptavidin using flow cytometry.

FIG. 15 shows 1×10⁷ conidia/ml of heat-inactivated conidia from Rhizopus arrhizus incubated with a 30 amino acid MASP-1 derived peptide tagged with a biotin tag compared to a control comprising no peptide. The biotin tag was detected with a FITC-conjugated streptavidin using flow cytometry.

FIG. 16 shows binding data for 5 μg/ml and 10 μg/ml solutions of a MASP-1 derived 30 amino acid peptide that was expected to bind when incubated together with Aspergillus fumigatus. The peptide was tagged with a biotin tag and detected with FITC-conjugated streptavidin using flow cytometry.

FIG. 17 shows binding data for 5 μg/ml and 10 μg/ml solutions of a peptide from the CUB1 domain of MASP-1 that was not expected to bind when incubated together with Aspergillus fumigatus. The peptide was tagged with a biotin tag and detected with FITC-conjugated streptavidin using flow cytometry.

FIG. 18 shows binding data for 5 μg/ml and 10 μg/ml solutions of a random peptide that was not expected to bind when incubated together with Aspergillus fumigatus. The peptide was tagged with a biotin tag and detected with FITC-conjugated streptavidin using flow cytometry.

FIG. 19 shows a generalized view of a peptibody construct. A) Fc-peptide fusion-protein (peptibody) containing two peptides and one Fc region. The peptibodies are constructed as immunoglobulins with an Fc region (blue) and a hinge region, but with the 30 amino acid MASP-1 binding sequence instead of the Fab region (orange). Overall, the Fc region has the potential to activate and direct the immune system to kill the pathogenic fungus and moreover it increases the half-life of the molecule. Peptibodies with Fc regions from e.g. other IgG subclasses (B) or other immunoglobulin classes are also relevant.

FIG. 20 shows data for binding of peptibody at a concentration of 5 μg/ml to different growth stages of A. fumigatus. Fluorescent microscopy shows binding of the peptibody to germ tubes (top panel) and hyphae (bottom panel). The peptibody was detected with rabbit anti-human IgG antibody and an Alexa fluor 488-coupled goat anti-rabbit antibody.

FIG. 21 shows data for binding of peptibody at a concentration of 10 μg/ml to different growth stages of A. fumigatus. Fluorescent microscopy shows binding of the peptibody to germ tubes (top panel) and hyphae (bottom panel). The peptibody was detected with rabbit anti-human IgG antibody and an Alexa fluor 488-coupled goat anti-rabbit antibody.

FIG. 22 shows binding data for different growth stages of A. fumigatus without the addition of peptibody. Fluorescent microscopy shows the A. fumigatus sample after addition of rabbit anti-human IgG antibody and an Alexa fluor 488-coupled goat anti-rabbit antibody.

FIG. 23 shows the survival curve from the in vivo pilot study. Immunosuppressed mice were infected at day 0, then treated with peptibody at different concentrations and followed for 14 days. The green colored lines represent the peptibody treatment groups and the grey shows the PBS control (6 mice per group). At day 8, all mice treated with PBS had died, whereas all mice in the 60 mg/kg peptibody treatment group survived until the end of the experiment at day 14. Mantel-Cox test: * . . . p<0.05, ** . . . p<0.01, *** . . . p<0.001, . . . p<0.0001.

FIG. 24 shows the fungal burden in BAL fluid. At the time of death/end of study, bronchoalveolar lavage (BAL) fluid was collected from the mouse lungs and cultured on agar plates to test the fungal survival in the lung cavity. In the two highest dose groups (6 and 60 mg/kg) the fungi were completely eradicated. Two mice per group were included as the lungs from the other mice were used for tissue analysis (FIG. 23).

FIG. 25 shows the fungal load in lung tissue. At the time of death/end of study, the lungs were homogenized and cultured on agar plates to test the fungal survival in the lung tissue. In the two highest dose groups (6 and 60 mg/kg) the fungi were eliminated as also seen in the BAL fluid in FIG. 7. The analysis was performed on four mice per group.

FIG. 26 shows binding of rMASP-1 wt and rMASP-1 mut to A. fumigatus. 5 μg/ml recombinant wild-type MASP-1 (⁴⁴⁴KLMAR⁴⁴⁸) or mutated rMASP-1 (⁴⁴⁴DDDDK⁴⁴⁸) were incubated with inact. A. fumigatus conidia and the binding of rMASP-1 was detected using an anti-MASP-1/-3/MAP-1 monoclonal antibody 8B3 followed by a FITC-conjugated secondary antibody. Binding was measured using flow cytometry.

FIG. 27 shows peptibody binding to live and inactivated A. fumigatus. Live A. fumigatus conidia or heat-inactivated conidia were incubated with 5 μg/ml peptibody and the binding was detected with an anti-human IgG polyclonal antibody and measured using flow cytometry.

FIG. 28 shows binding of mutated active and zymogen rMASP-1 to A. fumigatus. Mutated zymogen and activated rMASP-1 (⁴⁴⁴DDDDK⁴⁴⁸) and wild-type rMASP-1 (⁴⁴⁴KLMAR⁴⁴⁸) were incubated with inactivated A. fumigatus conidia and the binding of rMASP-1 was detected using an anti-MASP-1/-3/MAP-1 monoclonal antibody 8B3 followed by a FITC-conjugated secondary antibody. Binding was measured using flow cytometry.

FIG. 29 shows the binding of modified MASP-1 peptides to A. fumigatus as measured in terms of mean fluorescence intensity (MF/). Substituting a cysteine with a serine at amino acid position 7 (C7S) from the N-terminal does not affect binding. Substitution at position 11 (C11S) or 7+11 (C7S+C11S) decreases the binding.

FIG. 30 shows the ability of the MASP-1 peptide to bind A. fumigatus conidia with or without calcium. Peptide binding to A. fumigatus was detected using a biotin-tagged peptide and a FITC-coupled streptavidin. The MASP-1 peptide binds A. fumigatus both in the presence and absence of calcium.

FIG. 31 shows binding of the MASP-1 peptide and the corresponding MASP-3 peptide. The 30 aa MASP-3 peptide located at the same position as the MASP-1 peptide and having partly sequence overlap does not bind A. fumigatus as the MASP-1 peptide.

FIG. 32 shows binding of the MASP-1 and MASP-3 peptides to human cell lines. Neither of the 30 aa MASP peptides bind to the THP-1 monocytes (FIG. 32 B) or the HMEC-1 endothelial cells (FIG. 32 A).

FIGS. 33-40 shows peptibody binding to the different A. fumigatus growth stages using the same microscope imaging parameters for all growth stages (with peptibody in FIGS. 33-36 vs no peptibody in FIGS. 37-40). The peptibody binds stronger to germ tubes (FIG. 35) and hyphae (FIG. 36) than conidia (FIGS. 33-34). Moreover, the Peptibody preferably binds to the hyphal tip, which is the growth zone of the fungus (FIG. 35-36). No binding was detected in samples that were not treated with peptibody (negative control; FIGS. 37-40).

FIG. 41 shows the ability of several peptide sequences of the MASP-1 CCP2 domain to bind A. fumigatus conidia in comparison d to the MASP-1 binding peptide (SEQ ID NO:1). None of the CCP2 domain peptide sequences (CCP2 Ctrl.Pep1-3) demonstrated fungus binding capabilities as measured in terms of median fluorescence intensity (MFI).

### EXAMPLES

### Example 1—MASP-1 Binding

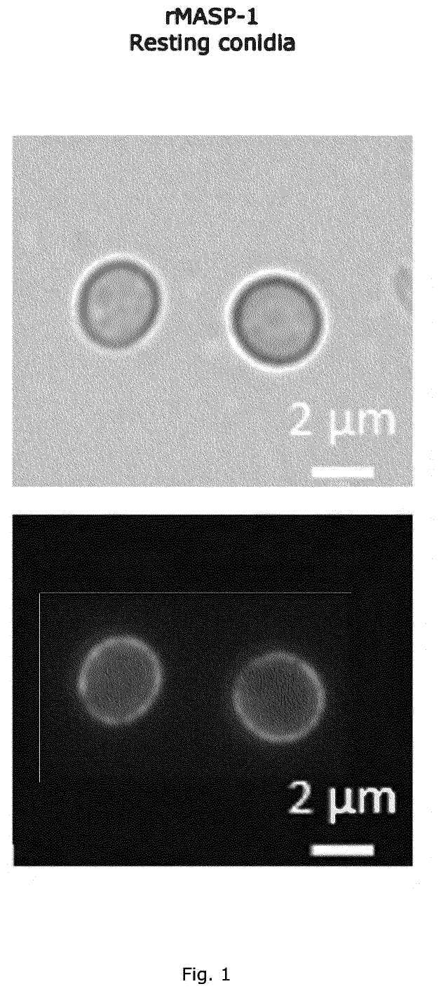

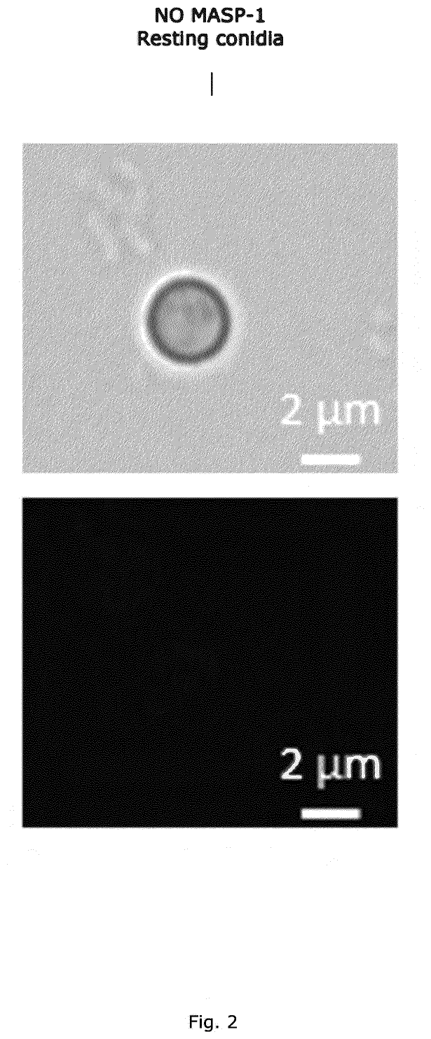

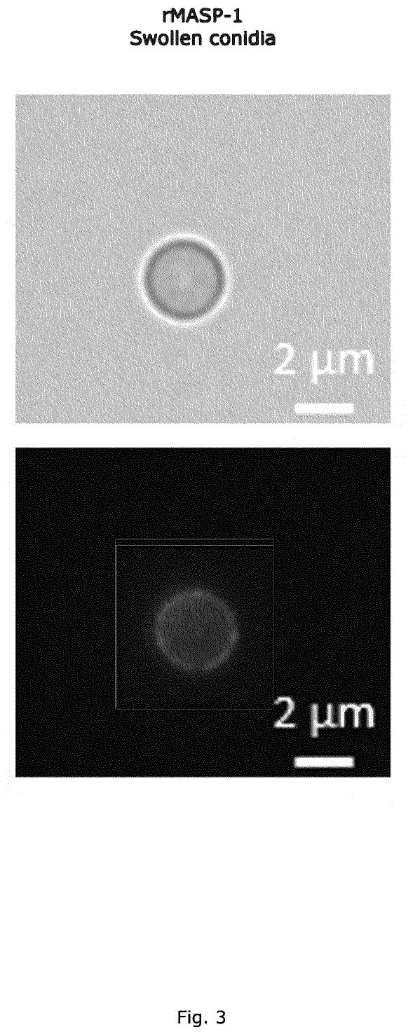

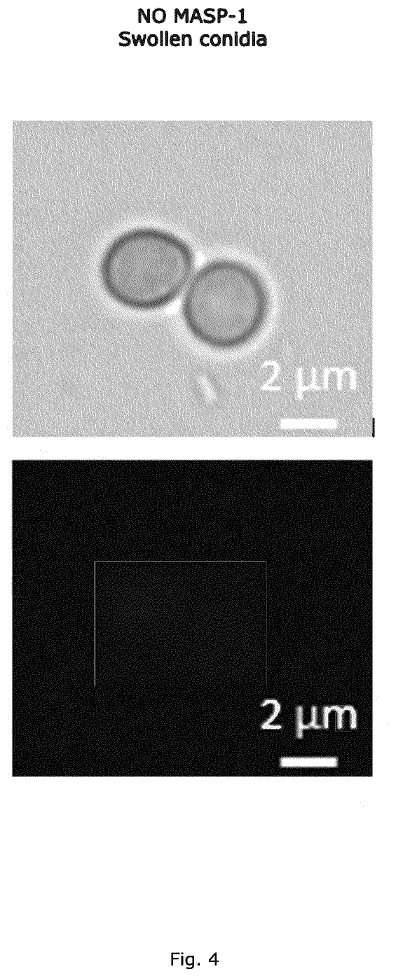

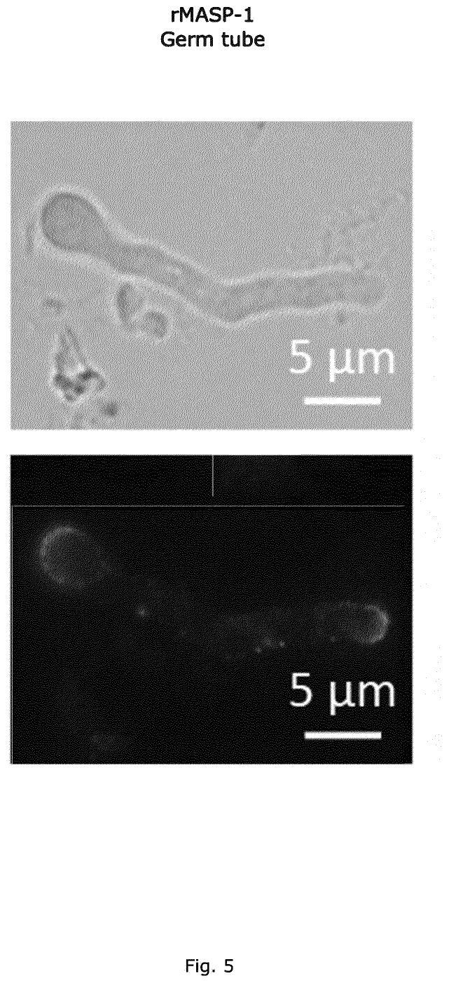

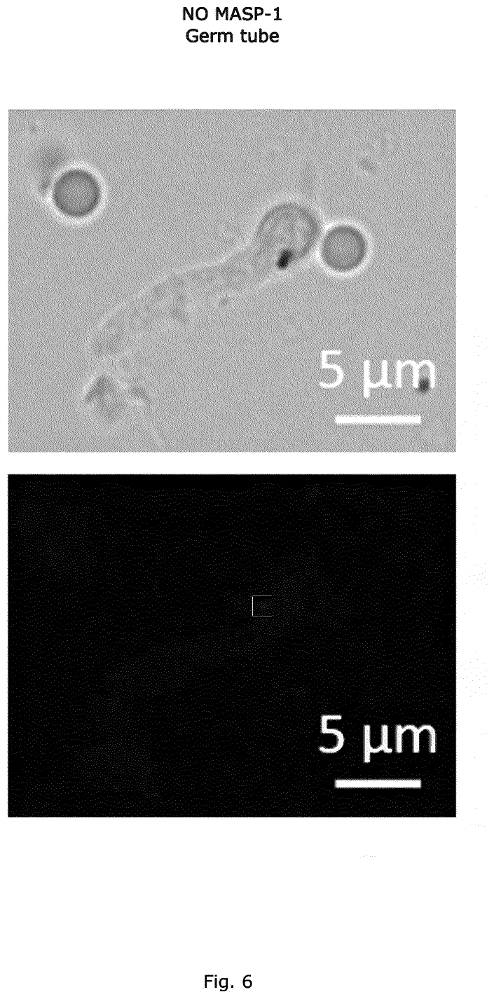

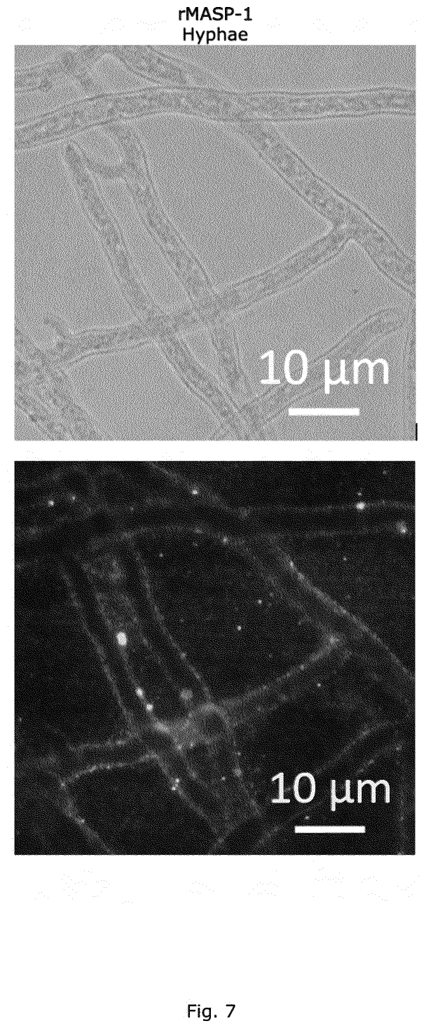

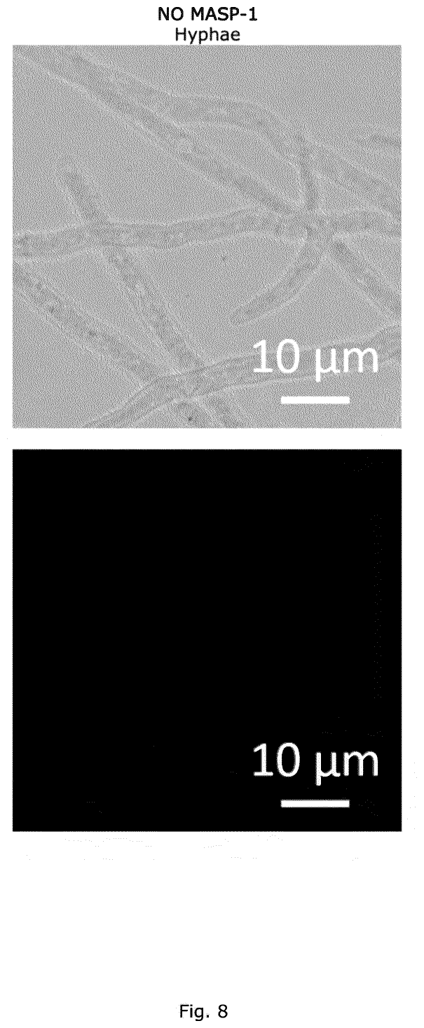

MASP-1, a serine protease from the complement system, binds directly to various pathogenic fungi, for example, Aspergillus fumigatus and this knowledge can be utilized to design a targeted antifungal compound. MASP-1 binding was tested on the different growth stages of A. fumigatus. First, the A. fumigatus conidia were incubated on microscopy glass slides for 0, 4, 8 and 16 hours to obtain resting conidia, swollen conidia, germ tubes and hyphae. Recombinant MASP-1 was then added in a concentration of 5 μg/ml and binding was detected with a pan anti-MASP-1/-3/MAP-1 monoclonal antibody 8B3 and Alexa fluor 488-coupled goat anti-mouse antibody. Using fluorescence microscopy, recombinant MASP-1 binding was detected for all growth stages (FIG. 1-8).

### Example 2—A 30 Amino Acid MASP-1 Peptide Binds to Opportunistic Fungi

### Background

Opportunistic fungi cause severe disease in immunocompromised patients and treatment can be challenging as mortality rates are high and treatment is further complicated by drug-drug interactions, adverse effects and resistance development. Therefore, it is important to create new targeted drugs against fungal infections. MASP-1, a serine protease from the complement system, binds directly to various pathogenic fungi e.g., it binds to all growth stages (resting and swollen conidia, germ tubes and hyphae) of Aspergillus fumigatus. The binding motif could be utilized in targeted treatment, however, the MASP-1 binding motif is not known.

### Methods

A. fumigatus conidia were heat-inactivated (15 min 121° C.) to eliminate contamination in the laboratory. 1×10⁷ conidia/ml were incubated with 5 μg/ml recombinant wild-type MASP-1 (⁴⁴⁴KLMAR⁴⁴⁸) or mutated rMASP-1 (⁴⁴⁴DDDDK⁴⁴⁸). The binding of MASP-1 was detected using 10 μg/ml of a pan anti-MASP-1/-3/MAP-1 monoclonal antibody 8B3 followed by a FITC-conjugated goat anti-mouse polyclonal antibody. Binding was measured as the mean fluorescence intensity using flow cytometry (Beckman Coulter, Gallios).

### Results

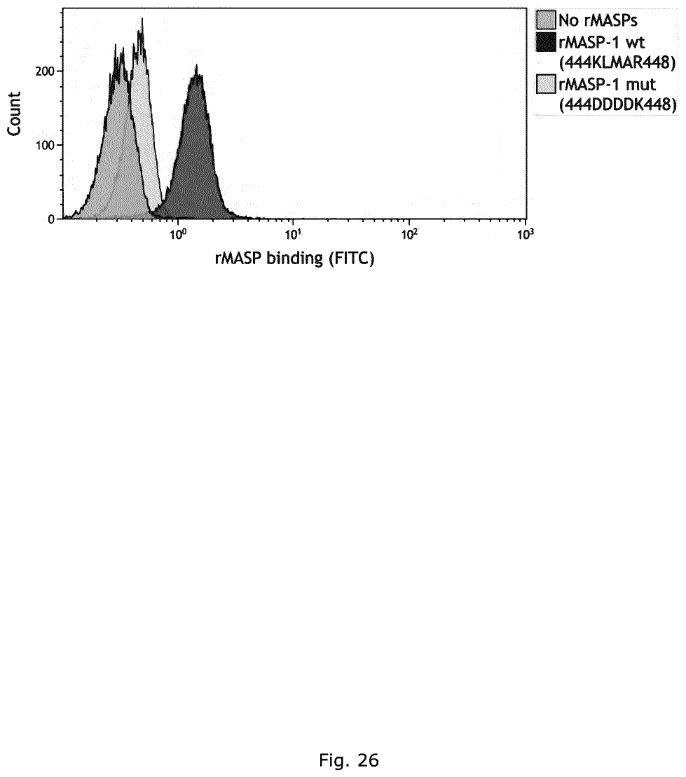

The study showed that wild-type rMASP-1 (⁴⁴⁴KLMAR⁴⁴⁸) binds to A. fumigatus conidia, whereas the mutated rMASP-1 (⁴⁴⁴DDDDK⁴⁴⁸) did not bind. Hence, the amino acid sequence KLMAR is necessary for the binding of MASP-1 to A. fumigatus as shown in FIG. 26.

### Example 2A—Zymogen Vs. Activated rMASP-1 (⁴⁴⁴DDDDK⁴⁴⁸) Binding to A. Fumigatus

Background The activated version of the mutated MASP-1 (⁴⁴⁴DDDDK⁴⁴⁸) does not bind to A. fumigatus. In the same experiment, it was also tested whether a zymogen version of the mutated rMASP-1 bound.

### Methods

The experiment in this example 2A was performed using the method as described in example 2.

### Results

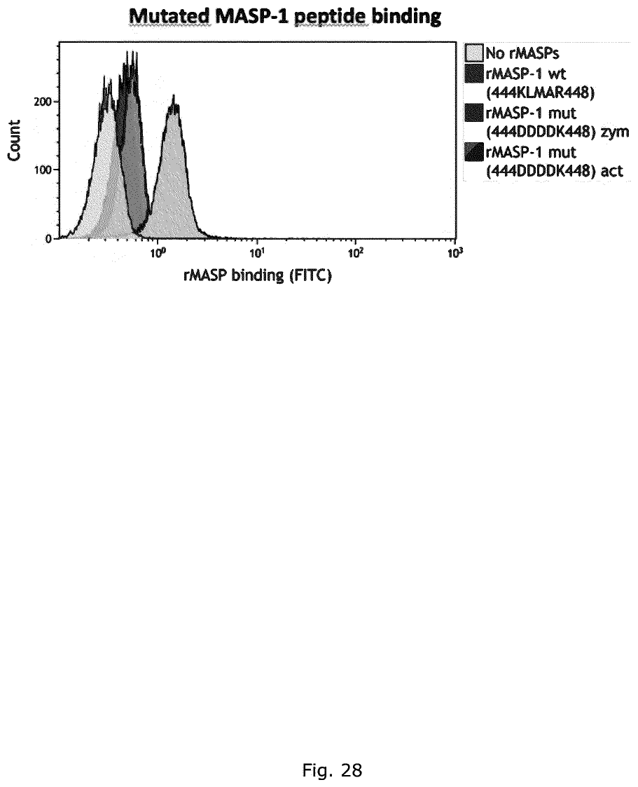

Neither the active nor zymogen mutated rMASP-1 (44⁴DDDDK⁴⁴⁸) bound to A. fumigatus conidia (FIG. 28). Hence, this confirms that the amino acid sequence KLMAR is crucial for binding MASP-1 to A. fumigatus.

### Example 3—A 30 Amino Acid MASP-1 Peptide Binds to Opportunistic Fungi

### Background

The MASP-1 amino acid sequence ⁴⁴⁴KLMAR⁴⁴⁸ is flanked by amino acids on both the N-terminal and C-terminal side of the protein and possibly an amino acid sequence specifically covering the KLMAR is capable of binding to opportunistic fungi.

### Methods

A 30 amino acid peptide comprising the KLMAR sequence was generated with an N-terminal biotin-tag. Besides, two control peptides were made; one with a random 30 amino acid sequence and one corresponding to a sequence from the MASP-1 CUB1 domain that does not contain the KLMAR sequence. 5 μg/ml of the N-term biotin-tagged peptides were incubated with 1×10⁷ heat-inactivated conidia/ml from different Aspergillus species and from fungi belonging to the Mucorales order. The peptide binding was subsequently detected with a FITC-coupled streptavidin using flow cytometry (Beckman Coulter, Gallios).

### Results

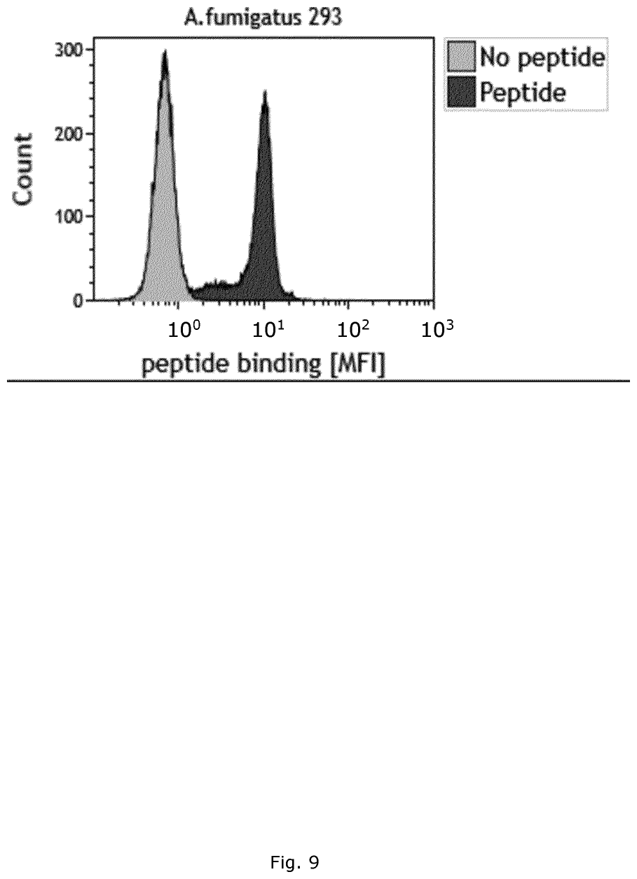

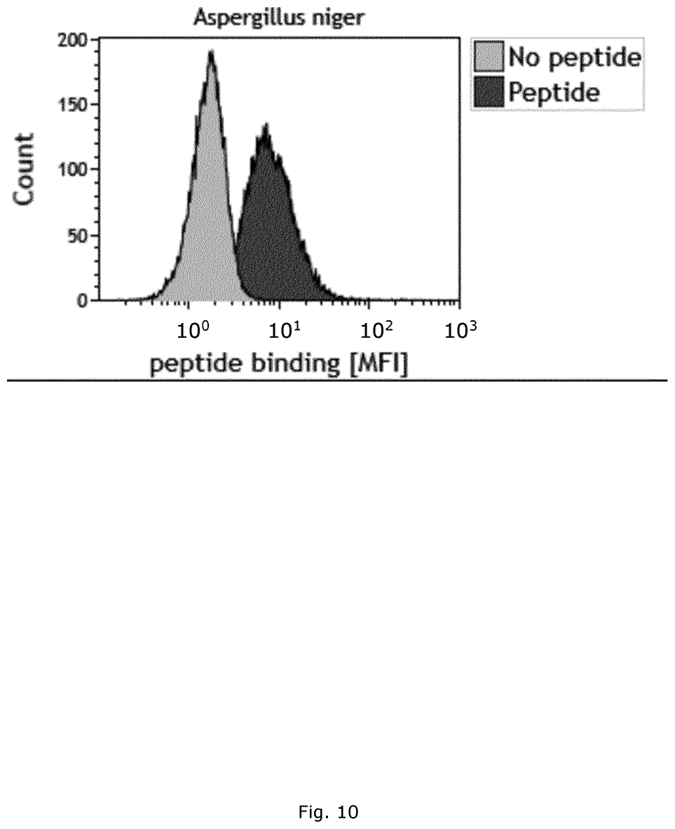

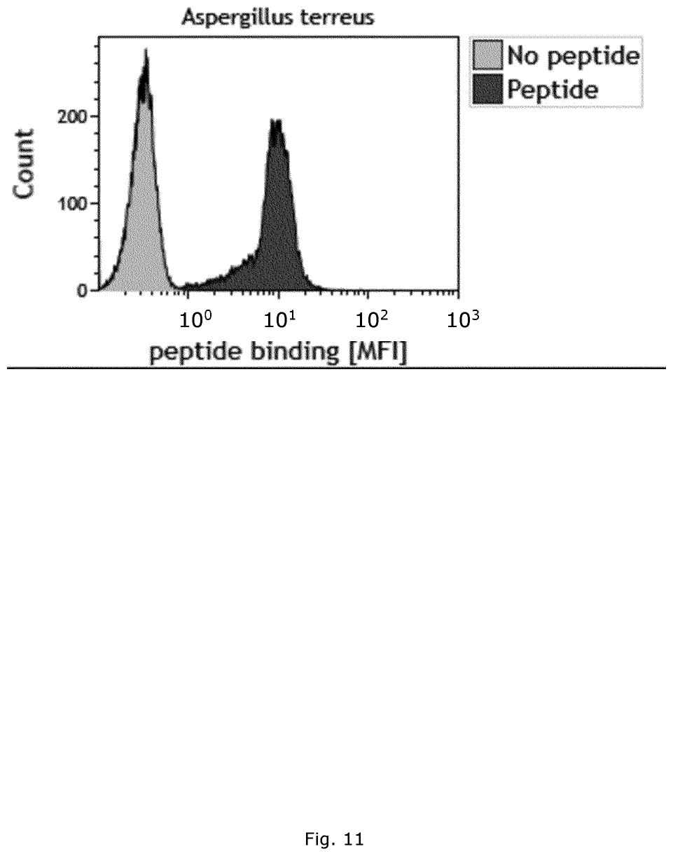

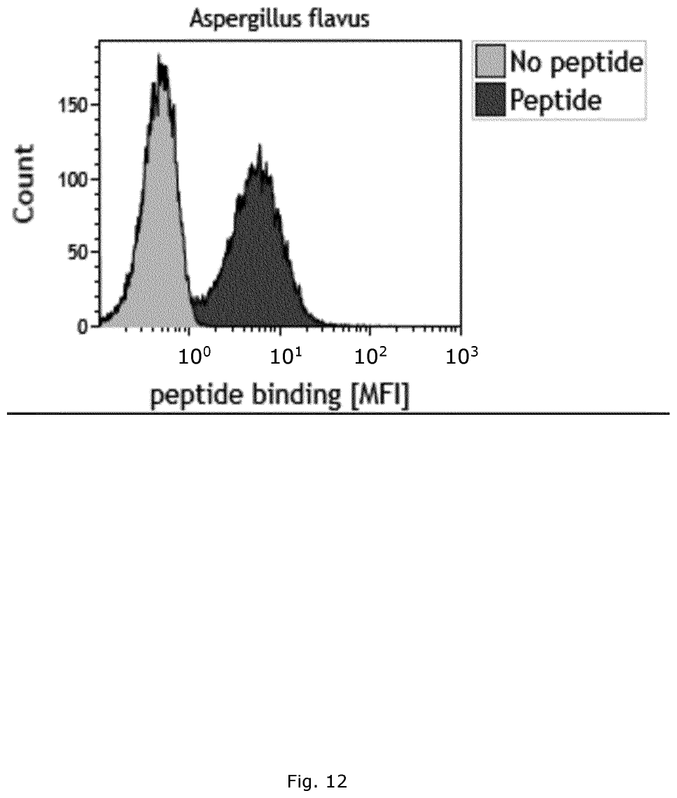

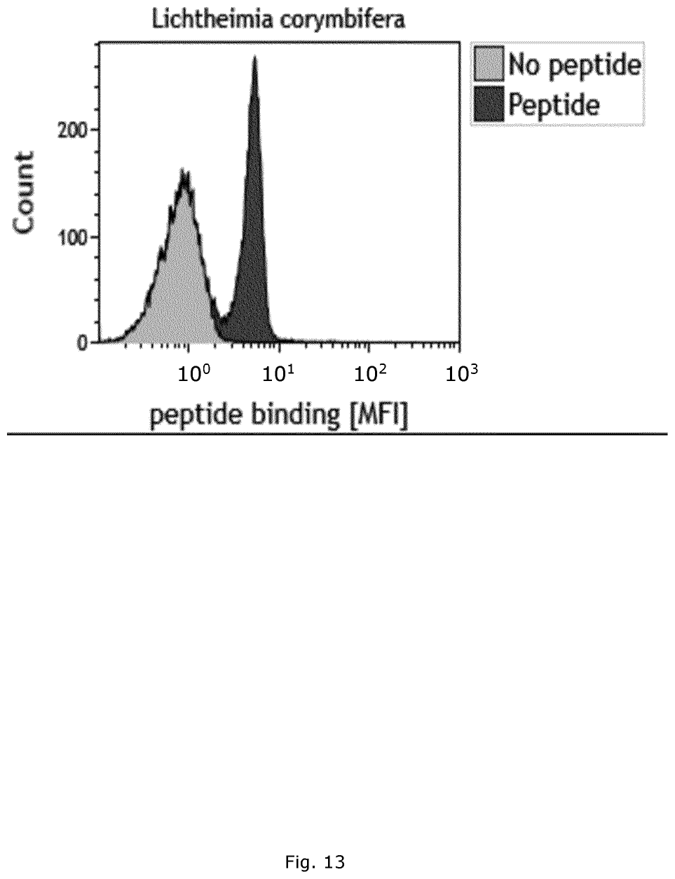

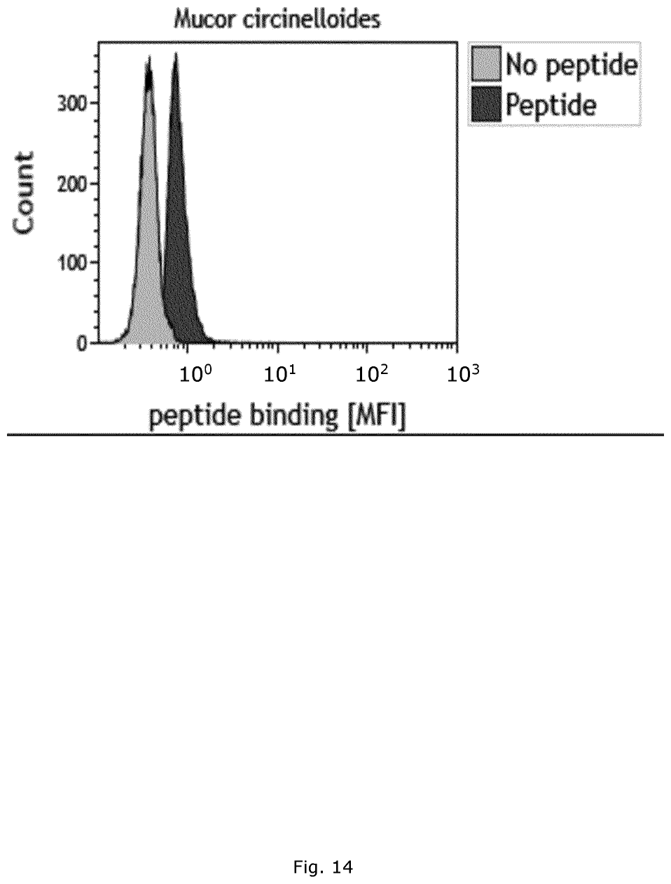

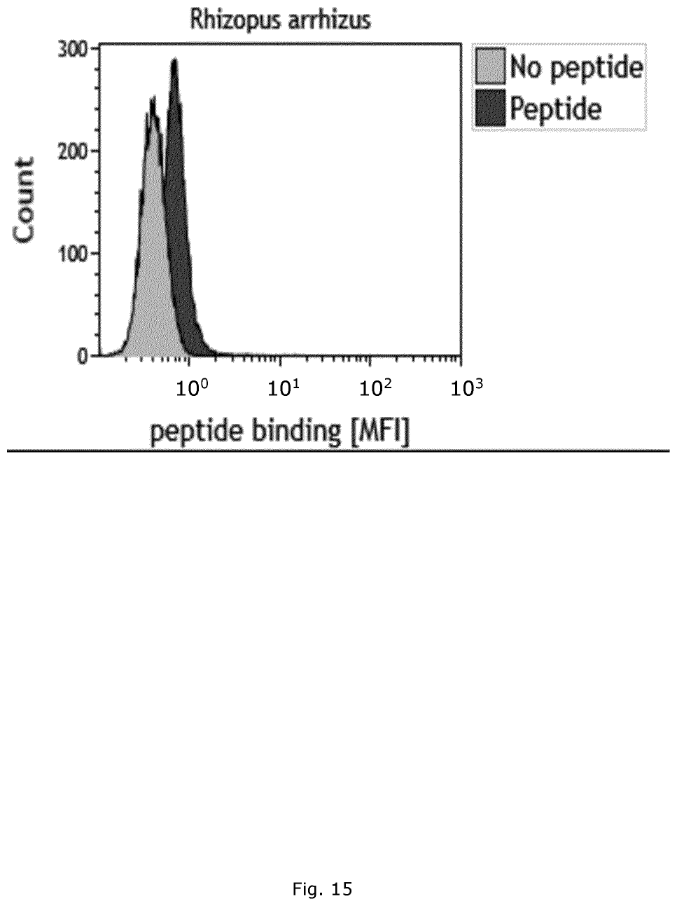

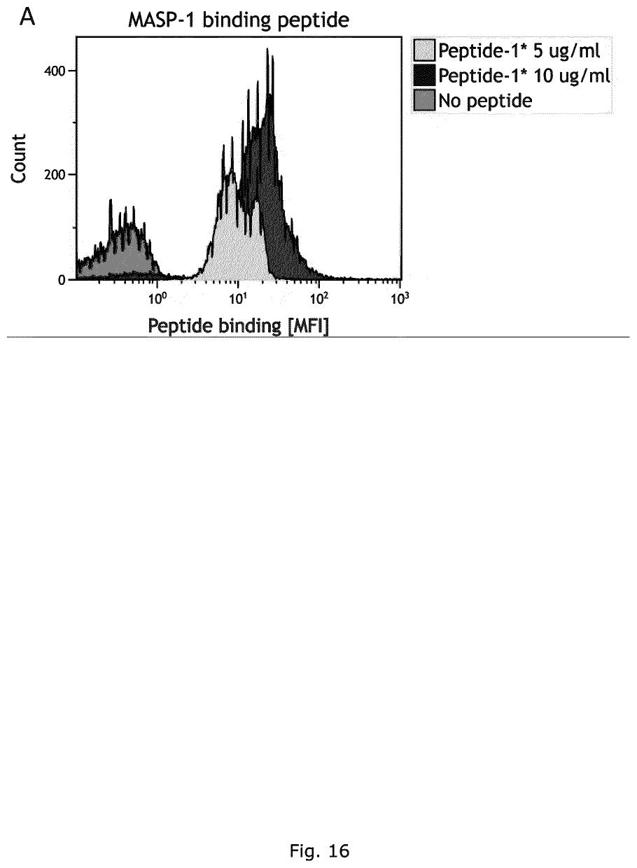

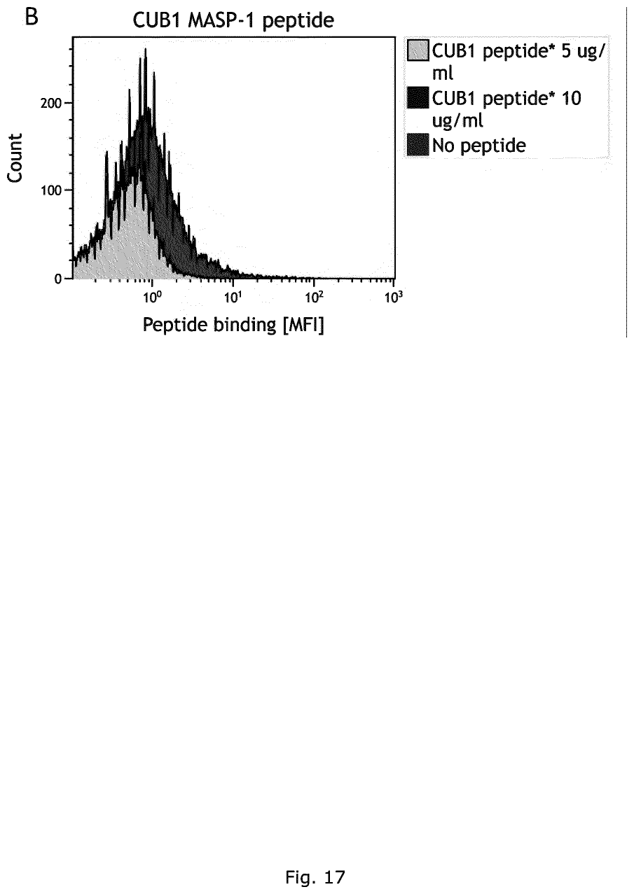

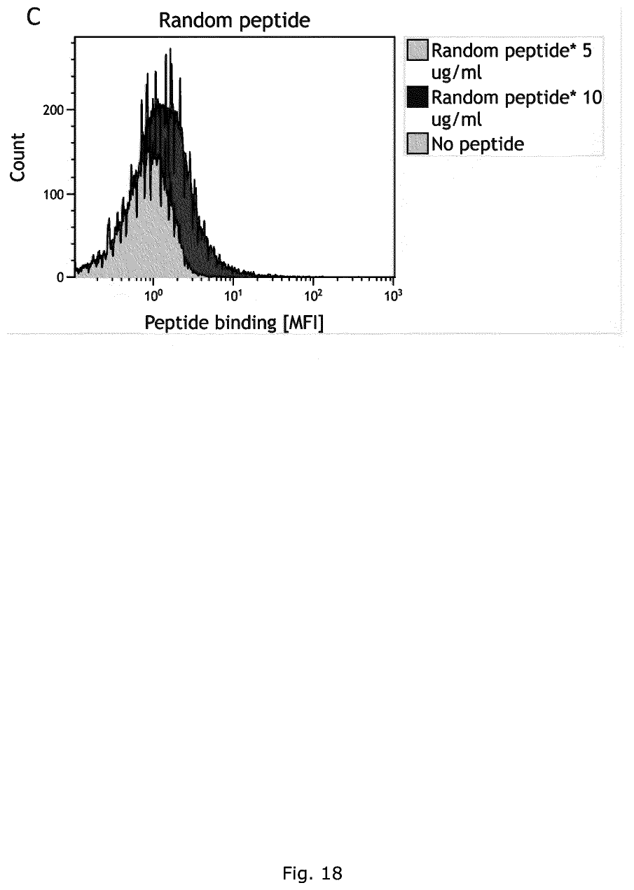

The KLMAR-comprising MASP-1 peptide successfully bound to the different Aspergillus (A. fumigatus, A. niger, A. terreus and A. flavus) and Mucorales fungi Lichtemia corymbifera, Mucor circinelloides and Rhizopus arrhizus (FIGS. 9-16) and the binding was specific for this peptide as A. fumigatus did not bind the random 30 amino peptide or the 30 amino acid MASP-1 CUB1 domain peptide (FIGS. 17-18).

### Example 3A

### Background

The 30 aa MASP-1 peptide binding to A. fumigatus is partly covering the CCP-2 domain of MASP-1. Hence, it was tested whether other 30 aa peptides from the same CCP-2 domain of MASP-1 would also bind to A. fumigatus.

### Methods

Three 30 amino acid peptides from the CCP2 domain of MASP-1 were generated with an N-terminal biotin-tag. CCP2 Ctrl.Pep1: VDCRAPGELEHGLITFSTRNNLTTYKSEIK, CCP2 Ctrl.Pep2: STRNNLTTYKSEIKYSCQEPYYKMLNNNTG, CCP2 Ctrl.Pep3: GELEHGLITFSTRNNLTTYKSEIKYSCQEP. 5 and 10 μg/ml of the N-term biotin-tagged peptides were incubated with 1×10⁷ heat-inactivated A. fumigatus conidia/ml and the peptide binding was detected with a FITC-coupled streptavidin using flow cytometry (BD, Celesta).

### Results

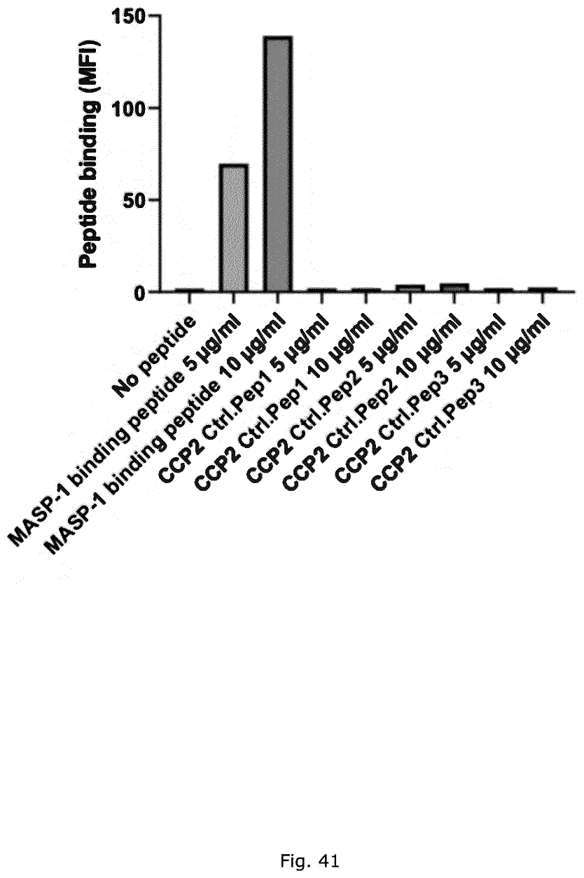

The three CCP2 peptides named CCP2 Ctrl. Pep1-3 did not bind to A. fumigatus conidia (FIG. 41). The KLMAR-comprising MASP-1 peptide (SEQ ID NO: 1) bound A. fumigatus as previously shown. Hence, the binding is specific to this peptide and does not involve the CCP2 domain in general.

### Example 3B—Binding of Modified MASP-1 Peptides

### Background

The 30 amino acid MASP-1 peptide has two cysteines at positions 7 and 11 from the N-terminal. Cysteines have the potential to form disulfide bridges which affect the tertiary structure of the protein. The question is whether the MASP-1 peptide binding depends on a disulfide bridge between the two cysteines.

### Methods

5 μg/ml of the N-term biotin-tagged peptides with C→S mutations at position 7 (C7S), position 11 (C11S) and both positions (C7S+C11S) were incubated with 1×10⁷ heat-inactivated A. fumigatus conidia/ml. The peptide binding was subsequently detected with a FITC-coupled streptavidin using flow cytometry (BD, Celesta).

### Results

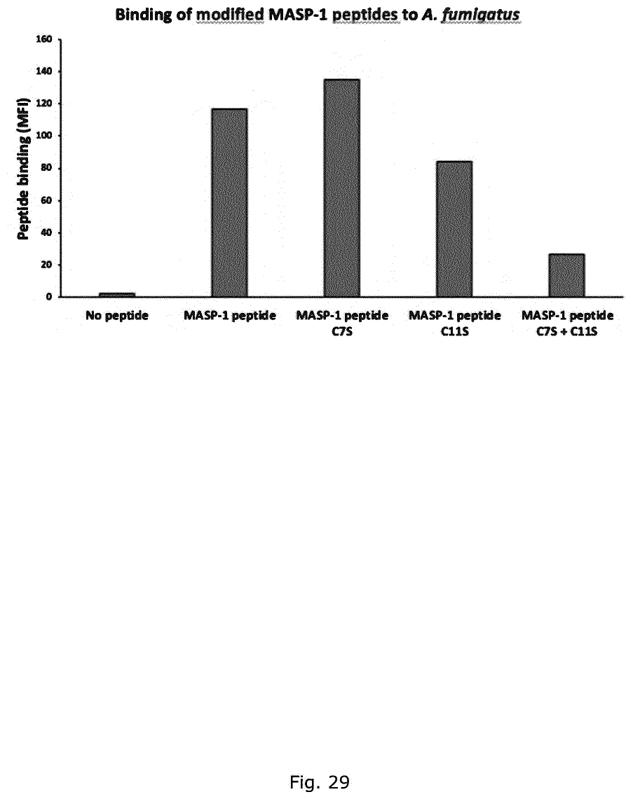

The peptide C7S binds in the same manner as the wild-type peptide. Hence the MASP-1 peptide does not seem to be dependent on a tertiary structure mediated by the cysteines as a mutation in only one of the cysteines should hinder disulfide bond formation. To our surprise, the cysteines still seem important for the binding as C11S and C7S+C11S bind less to the fungus (FIG. 29). The cysteines are located outside the ⁴⁴⁴KLMAR⁴⁴⁸sequence and thus have an unforeseen impact on the binding.

### Example 3C—Binding of the MASP-1 Peptide with or without Calcium

### Background

The binding of MASP-1 to other proteins like collectins and ficolins is calcium-dependent. Also, the dimerization of MASPs is calcium-dependent. It is unknown whether the MASP-1 peptide binding to A. fumigatus depends on calcium.

### Methods

5 μg/ml of the N-term biotin-tagged MASP-1 peptide were incubated with 1×10⁷ heat-inactivated A. fumigatus conidia/ml in barbital buffer (containing Ca2+) with or without 20 mM EDTA that chelates the calcium. The peptide binding was subsequently detected with a FITC-coupled streptavidin using flow cytometry (Beckman Coulter, Gallios).

### Results

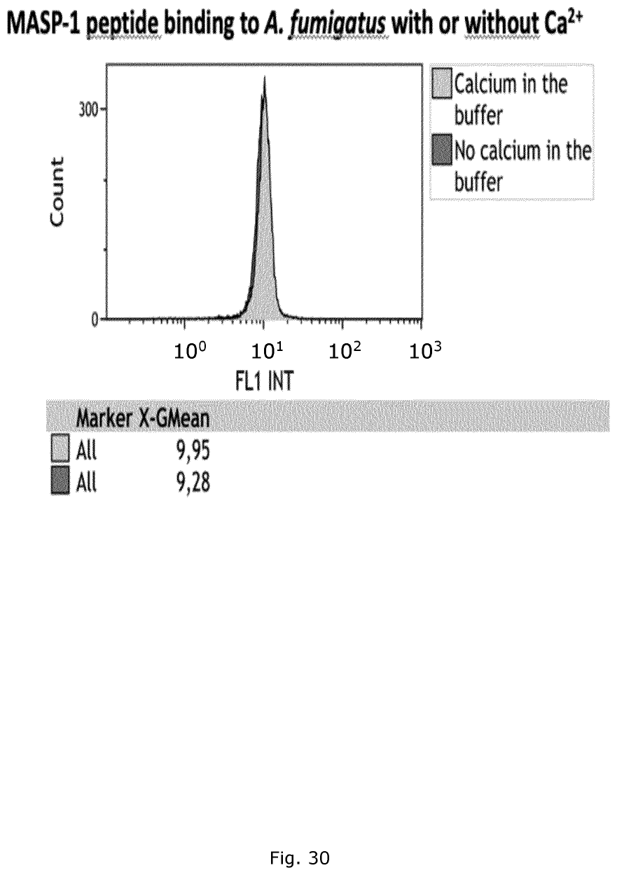

The MASP-1 peptide bound equally well to A. fumigatus with and without 20 mM EDTA. Hence, unlike the interaction with its binding partners from the immune system, MASP-1 does not require calcium for interacting with A. fumigatus (FIG. 30).

### Example 3D—Corresponding MASP-3 Peptide does not Bind A. fumigatus

### Background

MASP-1 and MASP-3 are splice variants of the common MASP1 gene and they both bind to A. fumigatus. The two serine proteases have the exact same heavy chain. Hence, it is expected that the two proteins bind to fungi with the same residues. In fact, it would seem unlikely if two nearly identical proteins binding to the same microbe do not share binding domains.

### Methods

The binding of the MASP-1 peptide was compared to the binding of a MASP-3 peptide covering the same protein region and thus having partly overlapping amino acid sequences. MASP-1 peptide: GRSLPTCLPVCGLPKFSRKLMARIFNGRPA (SEQ ID NO: 1). MASP-3 peptide: GRSLPTCLPECGQPSRSLPSLVKRIIGGRN (SEQ ID NO: 3). 5 μg/ml of the N-term biotin-tagged peptides were incubated with 1×10⁷ heat-inactivated A. fumigatus conidia/ml. The peptide binding was subsequently detected with a FITC-coupled streptavidin using flow cytometry (Beckman Coulter, Gallios).

### Results

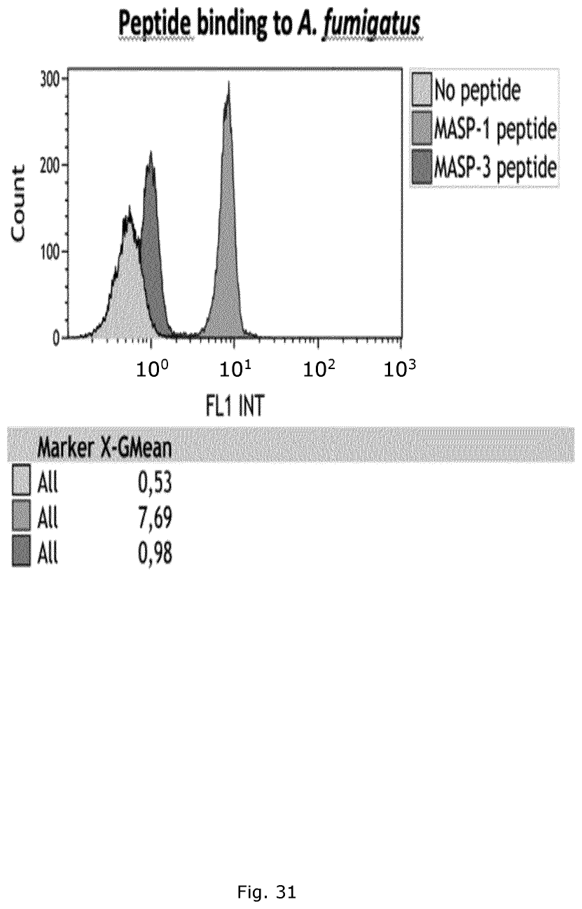

The MASP-3 peptide did not bind to A. fumigatus as the MASP-1 peptide (FIG. 31). So, despite the high degree of similarity between the two proteases, they have developed unequal ways of binding to fungi and the binding sequence thus is highly unpredictable.

### Example 3E—the MASP-1 Peptide does not Bind to Human Cells

### Background

The 30 aa MASP-1 peptide binds to various fungi, which makes it suitable for targeted treatment. It is, however, important that targeted antifungal treatment is specifically targeted towards fungi and not to human cells, as this increases the risk of unwanted drug effects. Therefore, it is convenient to test the binding of the MASP-1 peptide to human cells.

### Methods

5 μg/ml of the N-term biotin-tagged MASP-1 peptide was incubated with THP-1 and HMEC-1 cells (1×10⁷ cells/ml). The peptide binding was subsequently detected with a FITC-coupled streptavidin using flow cytometry (Beckman Coulter, Gallios).

### Results

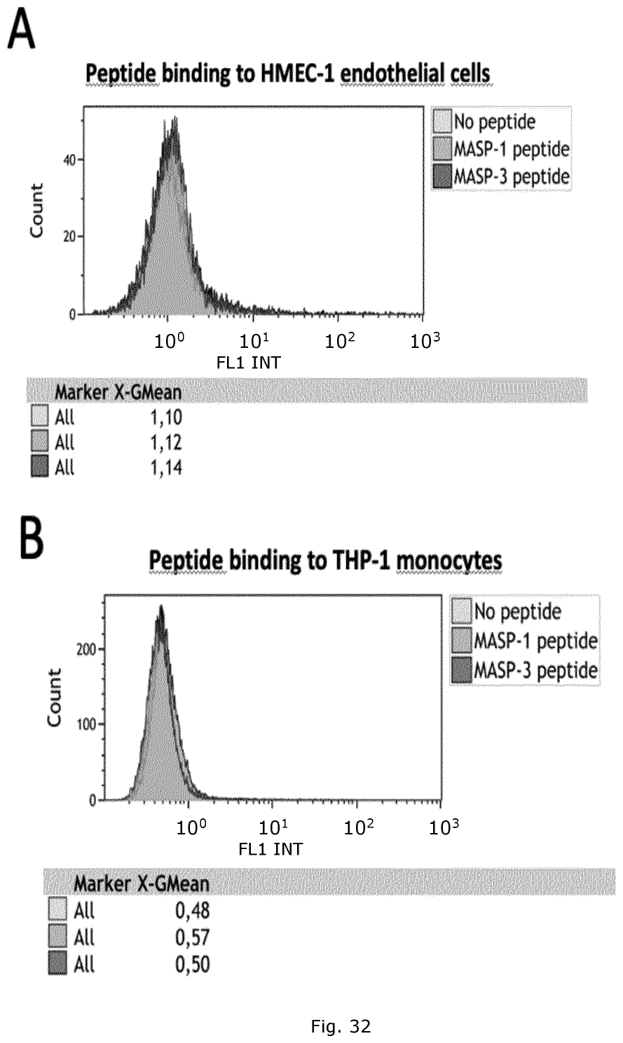

The monocytic cell line THP-1 (FIG. 32 B) and the endothelial cell line HMEC-1 (FIG. 32 A) did not show any binding to the MASP-1 peptide. Although binding to other cell types or primary cells cannot be ruled out, these results demonstrates that the peptide does not generally bind to human cells.

### Example 4—Peptibody Binding to A. fumigatus Conidia

### Background

A peptide-coupled functional component, e.g., an immunoglobulin Fc region, can work therapeutically against fungal infections by recruiting the immune apparatus of the patient, thereby facilitating clearance of the pathogen. An Fc region enables fungal killing via phagocytosis, antibody-dependent cellular cytotoxicity and by activating the complement system mediating further opsonization and phagocytosis as well as anaphylatoxin release and recruitment of inflammatory cells and complement-mediated cytolysis. Gathering multiple peptides with Fc regions also enables agglutination, which can block the fungi from accessing the epithelium and contribute to fungal clearance by making the phagocytosis more efficient. A peptide-coupled component can also work as a carrier of other therapeutic compounds such as antifungal agents e.g., azoles or as a carrier of radionuclides e.g., ²¹³Bi to destroy the invading fungi via local radiation.

A peptide-coupled component, e.g., an Fc region, can improve the pharmacokinetics and increase the in vivo half-life of the compound. An Fc region can also be manipulated to further increase the effectiveness and further prolong the half-life of the construct. An Fc region can for example be designed to be recycled in the endocytic pathway in host cells in order to increase the half-life of the construct. The compound can also be pegylated to extent the half-life and bioavailability.

The Fc region can be manipulated to change the mode of interaction with components from the immune system to increase/decrease the inflammatory process by e.g., changing the interaction with C1q from the complement system.

An Fc region can benefit the production of the construct by improving the structural and biochemical stability and by easing the purification process.

### Methods

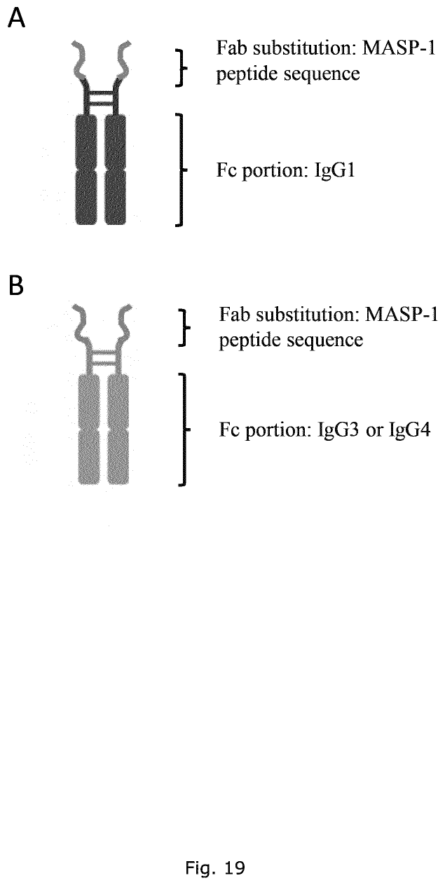

The first formulation made with the peptide is a fusion protein containing the peptide, a hinge region and the Fc-region of a human IgG1. This so-called Peptibody mimics the structure, function and biological half-life of natural immunoglobulins, but has the unique peptide binding region instead of the antigen-binding Fab fragment. An example of the structure of a peptibody construct is shown in FIG. 19. The Peptibody was produced recombinantly using Expi293 cells and purified using protein G Sepharose. Binding of the Peptibody to A. fumigatus conidia was tested by incubating 5 μg/ml of purified Peptibody with 1×10⁷ heat-inactivated or live conidia/ml and the binding was detected with a rabbit anti-human IgG antibody and a FITC-coupled goat anti-rabbit antibody using flow cytometry.

### Results

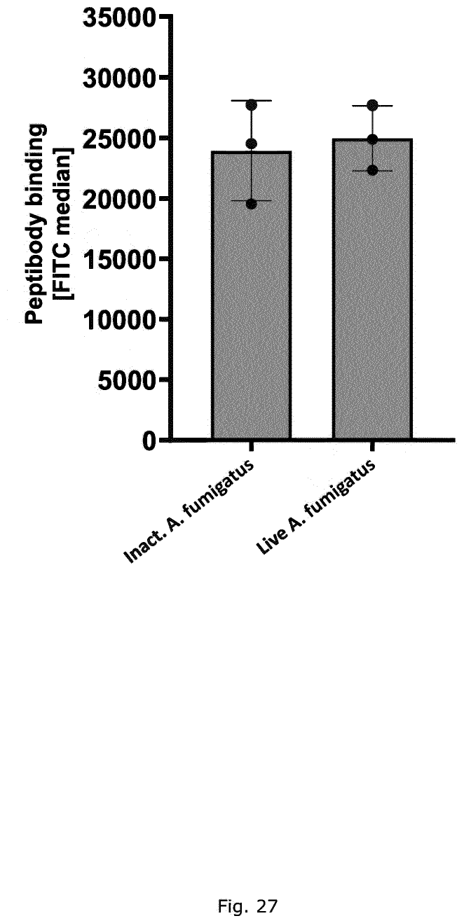

As shown in FIG. 27, the Peptibody binds to both the heat-inactivated and live A. fumigatus conidia.

### Example 5—Peptibody Binding to A. Fumigatus Germ Tubes and Hyphae

### Background

A. fumigatus is a filamentous fungus, which means that the conidia grow into elongated structures called germ tubes and hyphae. The fungus expands in its natural environment by growing hyphae structures and these growth stages are also found in infected patients. Thus, binding to these growth stages is likely an important feature of an antifungal drug in case the fungus has already developed in the patient prior to diagnosis and treatment.

### Methods

Binding of the Peptibody to germ tubes and hyphae was tested using fluorescence microscopy. First, the A. fumigatus conidia were incubated on microscopy glass slides for 6 and 18 hours to develop germ tubes and hyphae. Afterward, the Peptibody was added in a concentration of 5 or 10 μg/ml and binding was detected with rabbit anti-human IgG antibody and an Alexa fluor 488-coupled goat anti-rabbit antibody.

### Results

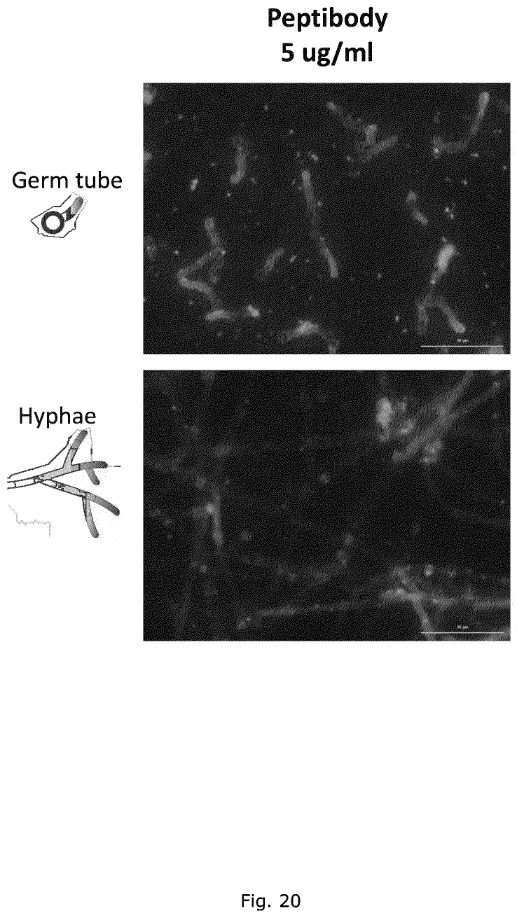

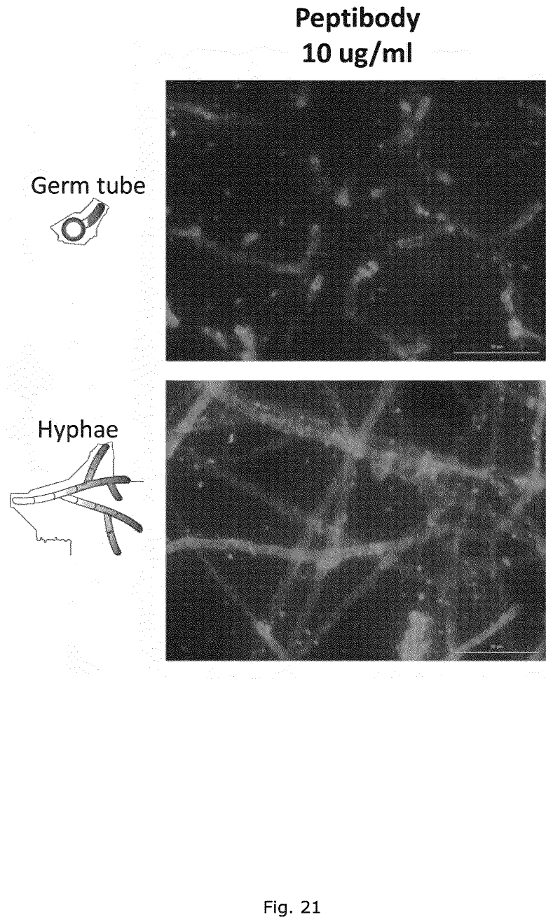

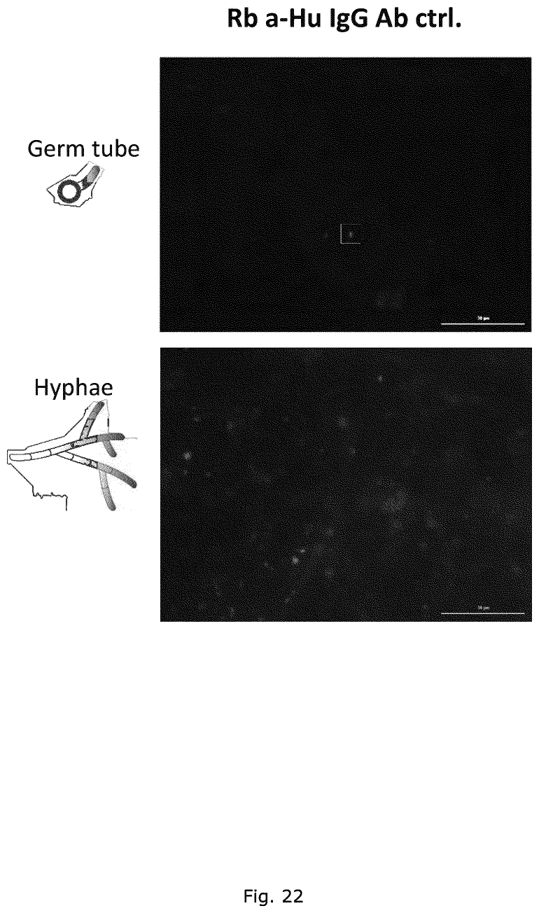

The Peptibody binding to A. fumigatus was visualized with fluorescence microscopy. A fluorescent signal appeared on A. fumigatus germ tubes and hyphae when the fungus was incubated with both 5 and 10 μg/ml Peptibody (FIGS. 20-21) and it did not appear when the Peptibody was omitted (FIG. 22). Hence, the Peptibody binds to A. fumigatus germ tubes and hyphae.

### Example 5A—Variation of Peptibody Binding to the Different Growth Stages

### Background

The cell wall of A. fumigatus changes composition during growth. Resting conidia have a resilient rodlet layer which is lost when the conidia get swollen and start elongating into germ tubes. Hence, the binding pattern of the Peptibody may change during growth.

### Methods

Comparison of the Peptibody binding to conidia, germ tubes and hyphae was made with fluorescence microscopy. A. fumigatus conidia were incubated on microscopy glass slides for 0, 2 6 and 17 hours to develop resting conidia, swollen conidia, germ tubes and hyphae, respectively. Afterward, the Peptibody was added in a concentration of 10 μg/ml and binding was detected with a rabbit anti-human IgG antibody and an Alexa fluor 488-coupled goat anti-rabbit antibody. The microscope imaging parameters were the same for all growth stages.

### Results

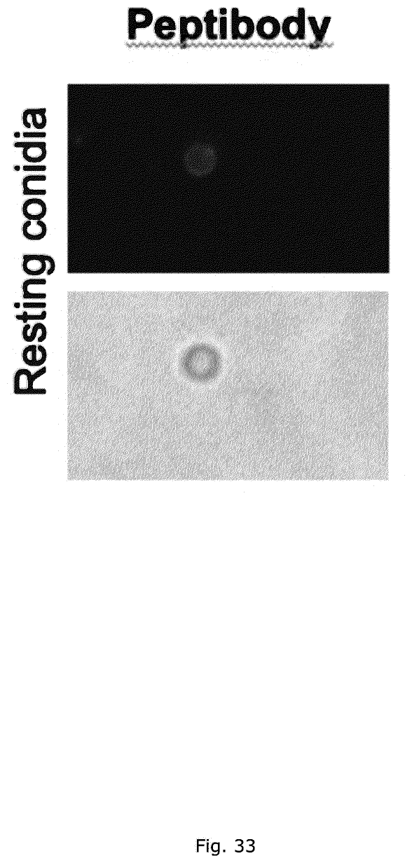

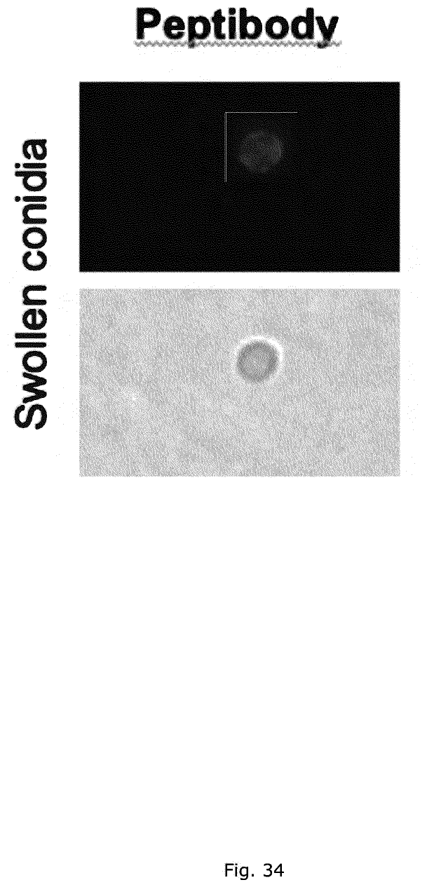

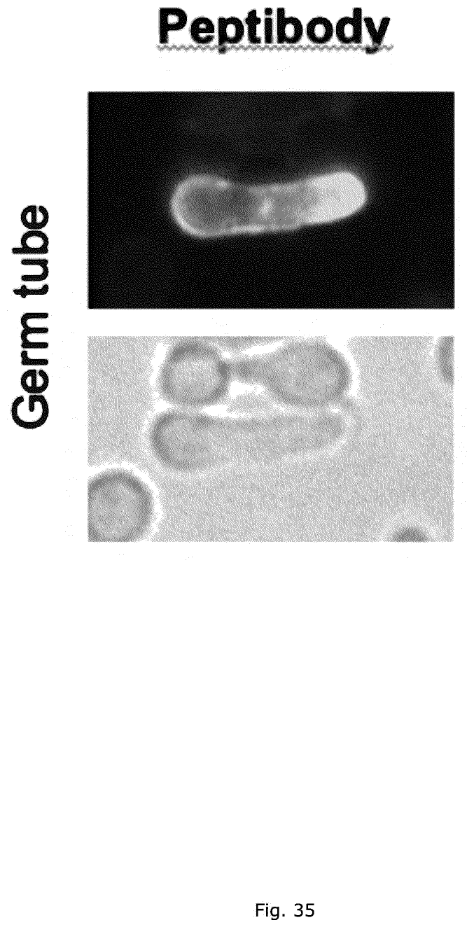

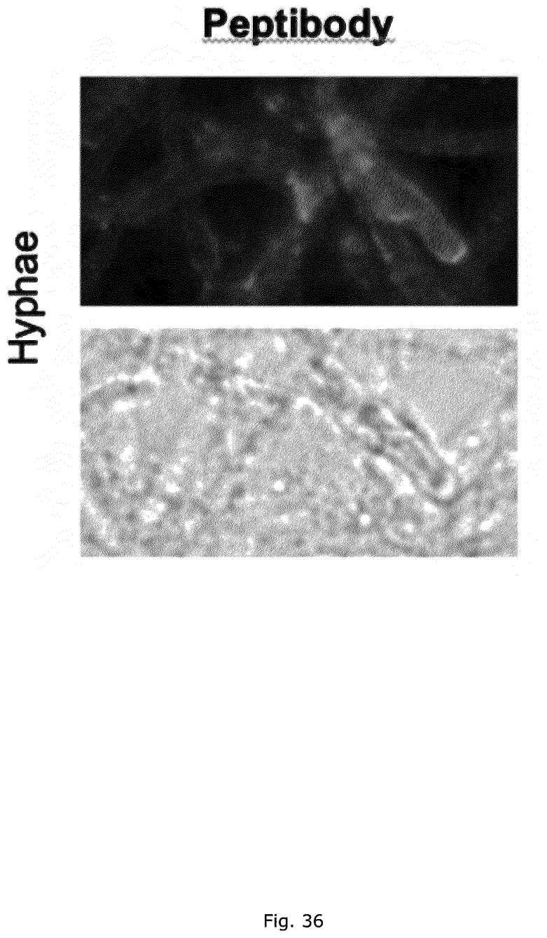

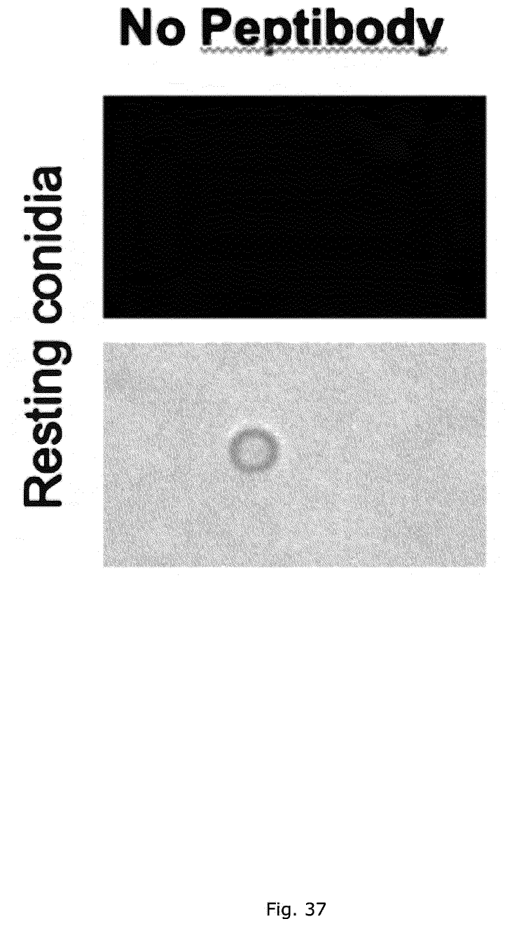

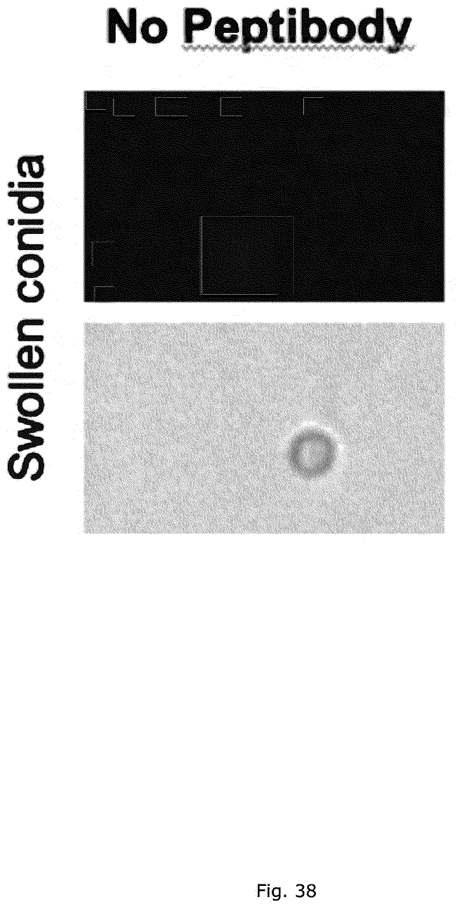

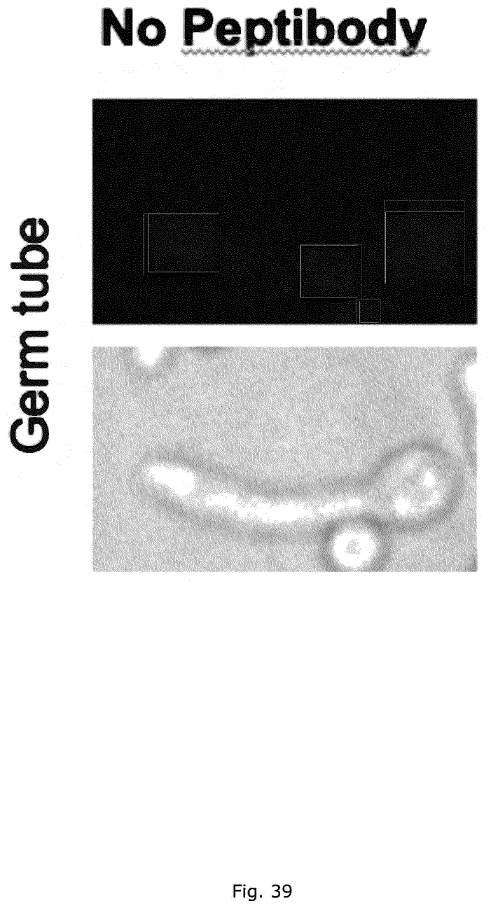

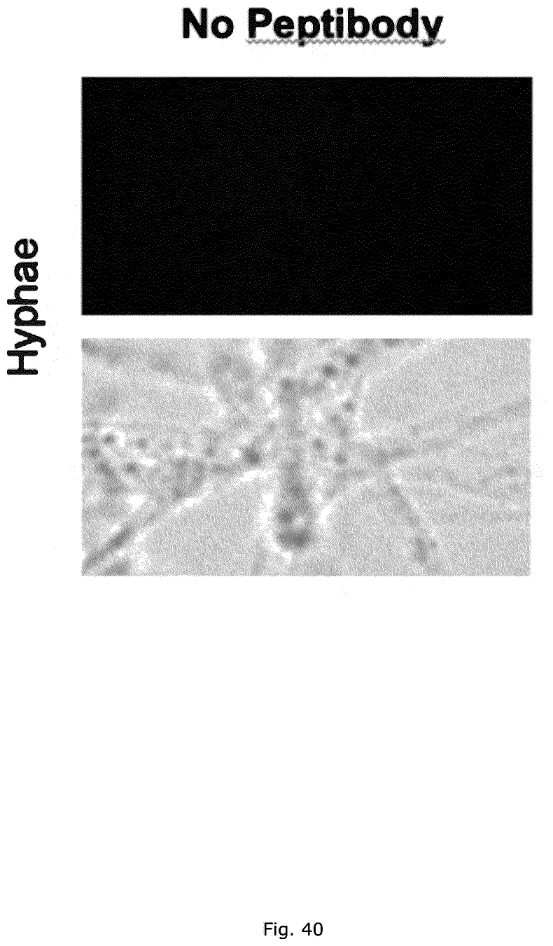

It seems that the binding capacity of the Peptibody to the different A. fumigatus growth stages vary. As illustrated in FIG. 33-40, the Peptibody binds stronger to germ tubes (FIG. 35) and hyphae (FIG. 36) than conidia. Moreover, the Peptibody preferably binds to the hyphal tip (FIG. 35-36), which is the growth zone of the fungus. This was surprising as the initial discovery was made on inactivated resting conidia and not on live growing fungi. The hyphal tip is likely a desired target for a therapeutic drug. Hence this Peptibody binding feature could be important for its therapeutic function.

### Example 6—Therapeutic Effect of the Peptibody in a Pre-Clinical Model

### Background

The Peptibody binds to A. fumigatus conidia, germ tubes and hyphae in vitro, however the in vivo effect of the Peptibody has to be tested in a pre-clinical animal model.

### Methods

The Peptibody was tested in a pre-clinical mouse model of an invasive A. fumigatus infection. C57BI/6J B6 mice were immunosuppressed with 150 mg/kg cyclophosphamide to mimic the immunocompromised human situation. The mice were then intranasally infected with 1×10⁷ A. fumigatus conidia (clinical strain A22) and treated intranasally with the Peptibody at different timepoints post-infection. Nasal injections were performed under mild anesthesia with isoflurane. The mice were divided in groups of 6 mice to test different dosages of Peptibody. The study period was 14 days, and the survival was monitored each day.

Cyclophosphamide and A. fumigatus injection schedule
Day −11: 150 mg/kg cyclophosphamide
Day −8: 150 mg/kg cyclophosphamide
Every 3^{rd} day: 100 mg/kg cyclophosphamide
Day 0: Intranasal A. fumigatus injection
Day 14: End of experiment

### Treatment Timepoints

1 hour post-infection
8 hours post-infection
24 hours post-infection

### Treatment Groups

P1) PBS
P2) 0.6 mg/kg
P3) 6 mg/kg
P4) 60 mg/kg

### Results

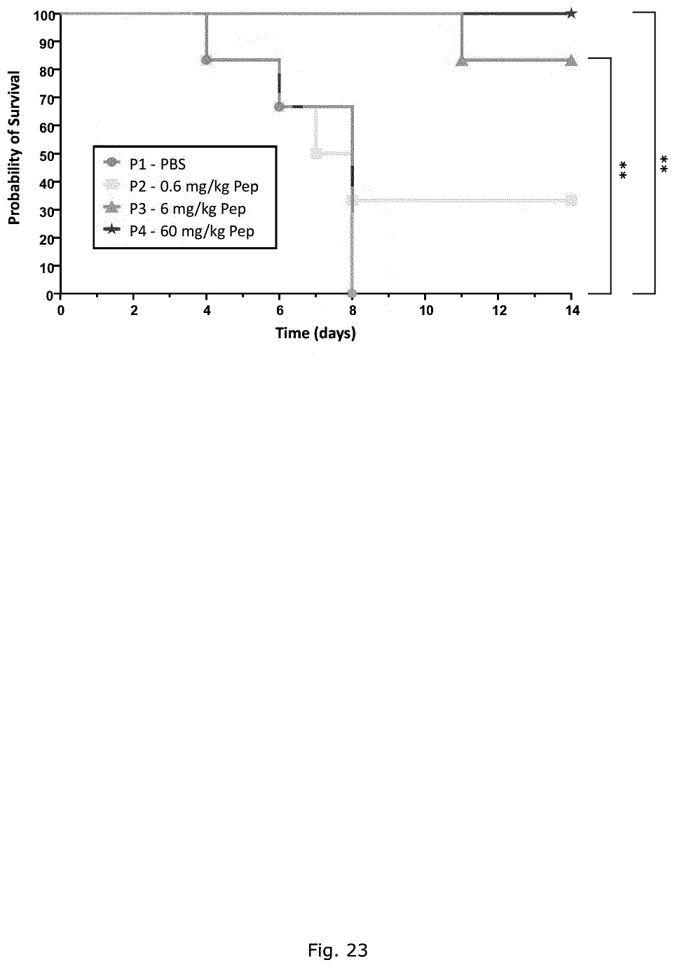

In the PBS group, all mice died within 8 days, whereas all mice survived the study period of 14 days with the highest Peptibody dose group P4 (60 mg/kg. Moreover, there was a dose-dependent effect of the Peptibody as the survival-percentage after 2 weeks was 33% in the P2 group (0.6 mg/kg) and 83% in the P3 group (6 mg/kg). Overall, it suggests that there is a therapeutic effect of the Peptibody against a pulmonary A. fumigatus infection. These results are shown in FIG. 23.

### Example 7—Peptibody Effect on the Fungal Burden in a Pre-Clinical Model

### Background

In the pre-clinical model described above, the A. fumigatus conidia were given intranasally, which means that the fungal infection starts in the lungs. It is therefore of interest to test whether the Peptibody relieves the fungal burden, meaning whether the Peptibody mediates killing of the fungi in the pulmonary cavities.

### Methods

Post-death (either due to the infection or due to the end of the study period), the lungs were flushed with PBS to collect the bronchoalveolar lavage fluid (BALF). The BALF was plated on Sabouraud Dextrose Agar in order to count the number of colony-forming units (CFU). In other mice from the treatment groups, the lungs were homogenized and plated on Sabouraud Dextrose Agar to count the number CFUs.

### Results

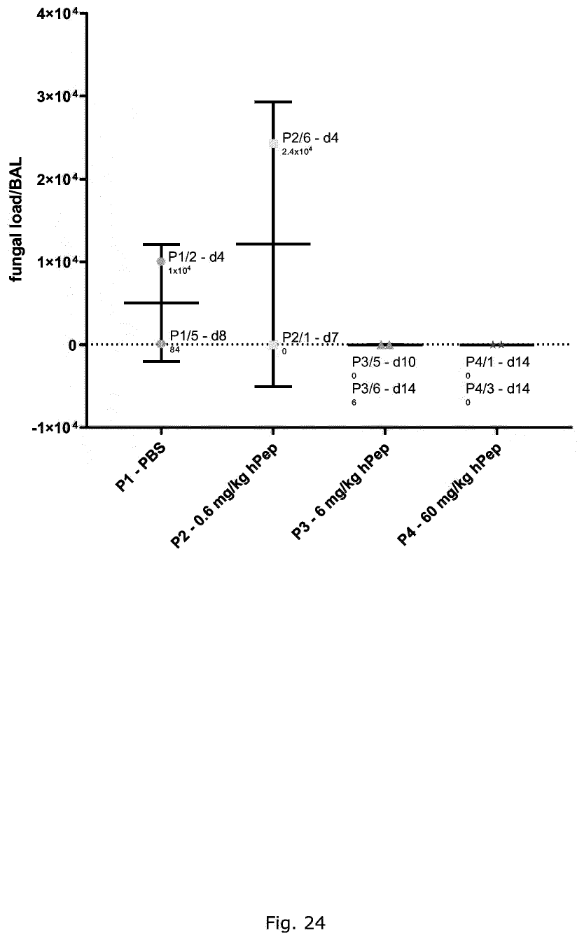

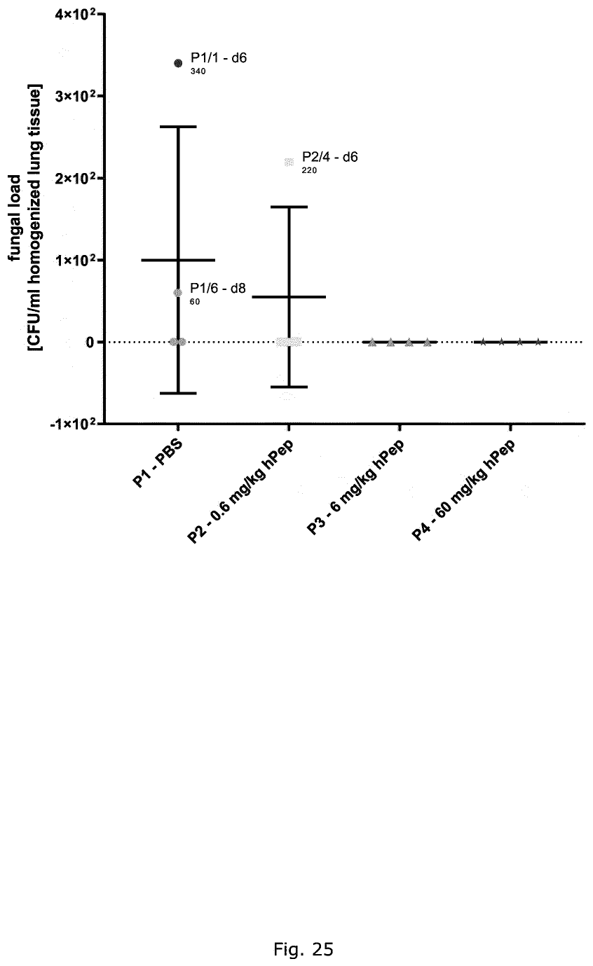

In both tests, the CFU count and thus the fungal burden, was eliminated in the P3 group (6 mg/kg) and the P4 (60 mg/kg) group as shown in FIG. 24-25. In these experiments, there were also a dose-dependent effect of the Peptibody since detectable CFUs was observed in the P2 group (0.6 mg/kg) and even more in the P1 (PBS) group. This suggests that the increased chance of survival mediated by the Peptibody is due to elimination of living fungi in the lungs.

SEQENCE LISTING

SEQ ID NO: 1
GRSLPTCLPVCGLPKFSRKLMARIFNGRPA
SEQ ID NO: 2
KLMAR
SEQ ID NO: 3-MASP-3 peptide
GRSLPTCLPECGQPSRSLPSLVKRIIGGRN
SEQ ID NO: 4 444-DDDDK-448 mutation
DDDDK
SEQ ID NO: 5-CCP2 Ctrl.Pep1
VDCRAPGELEHGLITFSTRNNLTTYKSEIK
SEQ ID NO: 6-CCP2 Ctrl.Pep2
STRNNLTTYKSEIKYSCQEPYYKMLNNNTG
SEQ ID NO: 7-CCP2 Ctrl.Pep3
GELEHGLITFSTRNNLTTYKSEIKYSCQEP

### Items

1: An isolated fungus binding amino acid comprising a binding part having at least 70% sequence identity to SEQ ID NO: 1
2. An isolated fungus binding amino acid comprising a binding motif having at least 80% sequence identity to SEQ ID NO: 2
3. An isolated fungus binding amino acid sequence according to item 2 being at least 10 amino acids long.
4. A fusion-protein construct that contains an amino acid sequence according to any of items 1-3 and the amino acid sequence of an Fc-region from a human immunoglobulin.
5. A fusion-protein construct according to item 4, wherein the human immunoglobulin is selected from the list consisting of an IgG1, IgG2, IgG3, IgG4, IgA1, IgA2, IgM, IgE, or IgD.
6. A fusion-protein construct according to item 4, wherein the human immunoglobulin is IgG1.
7. A fungus binding peptibody comprising a fungus binding amino acid conjugated to an Fc-region from a human immunoglobulin or a non-human immunoglobulin.
8. A fungus binding peptibody according to item 7, wherein the fungus binding amino acid sequence is according to any of claims 1-3.
9. A fungus binding peptibody according to any of items 7-8, wherein the peptibody further comprises a hinge region.
10. A fungus binding peptibody according to any of items 7-9, wherein the human immunoglobulin is selected from the list consisting of an IgG1, IgG2, IgG3, IgG4, IgA1, IgA2, IgM, IgE, or IgD.
11. A fungus binding peptibody according to item 10, wherein the human immunoglobulin is IgG1.
12. A fungus binding peptibody, wherein the structure is defined as in Formula (I)

$$X 1 - L 1 - F 1 \qquad Formula ( I )$$

wherein,
   X1 is a fungus binding amino acid according to any of claims 1-3,
   F1 is an Fc Region, and
   L1 is a peptide bond, linker or hinge region connecting X1 to F1
13. An isolated fungus binding amino acid sequence according to claims 1-3 or a fungus binding peptibody according to items 7-12 that is conjugated to an antibiotic.
14. An isolated fungus binding amino acid sequence according to claims 1-3 or a fungus binding peptibody according to items 7-12 that is PEGylated.
15. An isolated nucleic acid encoding a fungus binding amino acid sequence according to claim 1-3.
16. A nucleic acid construct encoding a fusion-protein according to claim 4-6.
17. An isolated nucleic acid comprising a nucleotide sequence which encodes a polypeptide according to any of items 1-12.
18. An isolated nucleic acid construct comprising an isolated nucleic acid according to claim 17 operably linked to one or more control sequences.
19. A host cell comprising a nucleic acid or nucleic acid construct according to claims 15-18.
20. A host cell according to claim 19, wherein said host cell expresses a fungus binding amino acid sequence encoded by a nucleic acid or nucleic acid construct according to items 15-18.
21. A method for producing a fungus binding amino acid sequence, the method comprising
   culturing the host cell according to item 20, and
   recovering the fungus binding amino acid sequence.
22. A composition comprising a fungus binding amino acid sequence according to any one of items 1-14.
23. A composition according to item 22 for use as a medicament.
24. A composition according to item 22 for use in therapy.
25. A composition according to item 22 for use in prophylaxis.
26. A composition according to item 22 for use in treating an immunocompromised individual.
27. A composition according to item 22 for use in treating an opportunistic fungal infection.
28. A composition according to item 27, wherein the opportunistic fungus belongs to the order of Mucorales.
29. A composition according to item 27, wherein the opportunistic fungus is a species belonging to the genus Aspergillus.
30. A composition according to item 22-29, wherein the composition is administrated nasal injection, subcutaneous injection, intravenous injection, inhalation or intratracheal injection.
31. A composition according to item 22 for use in diagnostics of a fungal infection.
32. A method for detecting a fungus in a sample comprising
   providing a sample suspected of containing a fungus
   adding a fungus binding amino acid according to any one of items 1-14 to the sample
   identifying the binding of the fungus binding amino acid to the fungus or fungal fragments within the sample.
33. A method according to item 32, where binding of the fungus binding amino acid to the sample is identified by immunodetection or a radionuclide or a signalling molecule.

1. An isolated fungus binding amino acid comprising a binding part having at least 90% sequence identity to SEQ ID NO: 1 and being at the most 30 amino acids long.

2. An The isolated fungus binding amino acid according to claim 1 comprising SEQ ID NO: 2.

3. The isolated fungus binding amino acid according to claim 1 being at least 10 amino acids long.

4-15. (canceled)

16. The isolated fungus binding amino acid sequence according to claim 1 being at least 20 amino acids long.

17. The isolated fungus binding amino acid according to claim 1 being SEQ ID NO: 1.

18. A fusion-protein construct that contains an amino acid sequence according to claim 1 and the amino acid sequence of an Fc-region from an immunoglobulin.

19. The isolated fungus binding amino acid according to claim 1, being coupled to a radionucleotide or a cytotoxic drug.

20. A peptibody comprising the fungus binding amino acid according to claim 1 conjugated to an Fc-region from a human immunoglobulin or a non-human immunoglobulin.

21. A peptibody comprising the fungus binding amino acid according to claim 1 conjugated to an Fc-region from a human immunoglobulin, wherein the human immunoglobulin is selected from the group consisting of an IgG1, IgG2, IgG3, IgG4, IgA1, IgA2, IgM, IgE, and IgD.

22. A method of treating or alleviating a fungus infection in a subject, the method comprising administering to the subject an isolated fungus binding amino acid comprising a binding part having at least 90% sequence identity to SEQ ID NO: 1 and being at the most 30 amino acids long.

23. The method according to claim 22, wherein the isolated fungus binding amino acid comprises SEQ ID NO: 2.

24. The method according to claim 22, wherein the isolated fungus binding amino acid being at least 10 amino acids long.

25. The method according to claim 22, wherein the isolated fungus binding amino acid being at least 20 amino acids long.

26. The method according to claim 22, wherein the isolated fungus binding amino acid being SEQ ID NO: 1.

27. The method according to claim 22, wherein the isolated fungus binding amino acid is in a fusion-protein construct that further comprises the amino acid sequence of an Fc-region from an immunoglobulin.

28. The method according to claim 22, wherein the isolated fungus binding amino acid being coupled to a radionucleotide or a cytotoxic drug.

29. The method according to claim 22, wherein the isolated fungus binding amino acid is in a peptibody that further comprises an Fc-region from a human immunoglobulin or a non-human immunoglobulin that is conjugated to the fungus binding amino acid.

30. The method according to claim 22, wherein the subject is immunocompromised.

31. The method according to claim 22, wherein the fungus infection is from a opportunistic fungal infection.

32. The method according to claim 22, wherein the fungus infection is pulmonary aspergillosis, cerebral aspergillosis, invasive aspergillosis, systemic aspergillosis, disseminated aspergillosis or aspergilloma.
